(19) United States
(12) Patent Application Publication
(10) Pub. No.: US 2021/0077292 A1
(43) Pub. Date: Mar. 18, 2021

(54) NASAL DILATION DEVICE AND METHOD OF USING

(71) Applicant: Schnozzle LLC, Baltimore, MD (US)

(72) Inventors: Clayton ANDREWS, Baltimore, MD (US); Patrick BYRNE, Baltimore, MD (US)

(21) Appl. No.: 16/612,140

(22) PCT Filed: May 8, 2018
(86) PCT No.: PCT/US18/31587

§ 371 Date: Nov. 8, 2019

Related U.S. Application Data
(60) Provisional application No. 62/503,269, filed on May 8, 2017.

(51) Int. Cl. A61F 5/08

(52) U.S. Cl. CPC A61F 5/08

(57) ABSTRACT

Provided are comfortable and discreet nasal dilator for the non-surgical management of nasal obstruction, wherein the dilator is configured to rest entirely within the nasal passage of a single nostril. Also provided are methods of using such dilators to relieve nasal obstruction and maximize airflow through the nostril.

### CROSS-REFERENCE TO RELATED APPLICATION

This application claims priority to U.S. Provisional Application No. 62/503,269 filed on May 8, 2017, the disclosure of which is incorporated herein by reference in its entirety.

### FIELD OF THE INVENTION

The present disclosure relates to a comfortable and discreet nasal dilator for the non-surgical management of nasal obstruction.

### BACKGROUND

One in four people regularly experience difficulty breathing through the nose. In fact, restricted nasal breathing is one of the most common complaints heard by otolaryngologists (ENTs) every day. These symptoms present a daily source of discomfort that reduces productivity and quality of life for those afflicted. Patients report a constant bother and source of embarrassment, difficulty sleeping and habitual snoring, forced reliance on mouth breathing, and limited stamina during physical activity.

Nasal obstruction affects millions of Americans, with up to 40% suffering from this at some point. The treatments available depend largely on the cause. Many patients suffer from inflammatory causes, as in the example of allergic rhinitis, wherein temporary tissue swelling occludes airflow. Other times, sufferers may experience these symptoms as a result of a deviated septum, narrow or collapsed nasal passages, and other structural problems that block or limit airflow. These anatomical sources of nasal obstruction affect over 40 million Americans and 12% of the population globally to a clinically significant degree. Further, many patients suffer from a combination of both inflammatory and anatomical causes.

A high percentage of patients have as the specific location of their obstruction the internal nasal valve. This is an anatomic and functional area approximately one centimeter beyond the nostril that tends to have the smallest cross-sectional area and offers the greatest resistance to airflow in the nasal cavity. This is exacerbated by the tendency of this area to collapse upon inspiration due to weak or damaged alar and lateral cartilage providing inadequate structural support. Therefore, treatments to assist in the dilation and/or support of the internal nasal valve tend to predominate for surgeons who care for these patients.

Currently, functional rhinoplasty and septoplasty are the leading treatments for nasal obstruction depending on the nature of the anatomic problem. This involves using cartilage grafts taken from the ears or ribs to permanently open the nasal passages and thereby decrease the resistance to airflow. However, positive results are not guaranteed, and after a painful, year long recovery, approximately 20% of patients return to their surgeons and report unimproved or worsened symptoms (FIG. 1). Moreover, many patients consider this option too extreme for their non-life-threatening symptoms.

Nasal strips and other over-the-counter nasal dilators better fit sufferers' preferences for treatment, as they instantly reproduce the effects of surgery without the risk. A number of such devices exist to curb snoring and improve sleep quality by relieving nasal obstruction. Most prevalent are the flexible strips that adhere to the surface of the nose and lift the lateral wall outward. While these devices are typically effective in relieving nasal obstruction, these devices are highly visible on the surface of the nose and challenging to adhere in the correct location. Moreover, users complain the adhesives cause skin irritation upon removal and often peel off prematurely during use due to sweaty or oily skin.

Many existing internal nasal dilators take the form of a pair of hollow cones or tubes connected by a thin strip of material. The user slides the tubes into each nostril and allows the strip connecting the tubes to clip onto the septum. These devices dilate the nasal valve by virtue of the size of the device. This class of nasal dilator is often semi-rigid and essentially serves as a stent within the nasal valve area, and the rigidity of these devices can make insertion and extraction uncomfortable and challenging. The inflexibility also requires manufacturers to make different sized devices to cater for a range of nasal passage sizes. The ring that clips onto the septum is also readily visible to onlookers and creates social stigma that makes use during the day unacceptable (FIG. 1).

Another significant issue with existing nasal dilators arises due to the paired, symmetric nature of nasal dilators. This problem is particularly relevant when the user has a deviated septum, which is a curvature in the cartilage and/or bone that separates the nostrils. A deviated septum causes the nasal passages to have different shapes and leads to airflow blockage through only one nostril. Most nasal dilators currently exist as symmetrical pairs, and in the case of septal deviation, if the dilator is of a sufficiently small size to enter one nostril, then the other identical dilator of the pair will be too small to be effective in dilating the other nostril. On the other hand, if one dilator of the symmetrical pair is sufficiently large to effectively dilate one nostril, its other half is too large to be inserted in the other nostril. Beyond the setting of septal deviation, nasal valve collapse by any cause can be asymmetric, leading to challenges using the currently available symmetric dilators. Overall, sizing is an important issue with existing intranasal dilators, as devices that are too small are not effective while devices that are too large stretch the nasal passage uncomfortably and cannot be tolerated for long-term use.

Currently, there is no nasal dilator that is simultaneously effective, discreet, comfortable, and highly usable. While the high degree of visibility of existing devices does not prevent use at night or during exercise, these solutions are not suitable for use throughout the day in personal and professional life due to the social stigma imposed by noticeably wearing a device inserted within the nose. Moreover, the discomfort imposed by existing devices is tolerable for short-term use during exercise and sleep; however, this discomfort becomes unbearable on the scale of entire days of continuous use.

Although the mechanism for reversing nasal obstruction is relatively straightforward, there is no viable alternative to surgery. As a result, millions of nasal obstruction sufferers currently forgo treatment. Faced with the choice between inaction and the appointments, extensive recovery, and uncertain outcome inherent with surgery, the majority of individuals—especially those with minor to moderate obstruction—elect to live with their symptoms, which present a constant bother, source of embarrassment, and long-term health risk. There is thus a need in the art for a comfortable and discreet nasal dilator for the non-surgical management of nasal obstruction.

### SUMMARY OF THE INVENTION

A preferred device is adjustable to fit most nasal cavities as well as each nasal cavity independently to dilate effectively and improve airflow (FIG. 2). The desirable nasal dilator is also comfortable, allowing users to wear the device for extended periods without pain. Moreover, a desirable nasal dilator should be completely discreet and rest entirely within the nasal passage without the use of external hooks, clips, or bands. A preferred device must also be designed for stability within the nasal passage to withstand normal physiological airflows and forces without dislodgment. Finally, the desirable nasal dilator must be easily inserted, extracted, and periodically adjusted within the nose, and it must be intuitive to place correctly within the nasal cavity for optimal results.

The present disclosure provides an internal nasal dilator that satisfies these needs and is designed to counteract nasal obstruction at its source—the internal nasal valve—to decrease the resistance to airflow and facilitate breathing. The device provides a dilation force that optimizes the tradeoff between efficacy and comfort. Furthermore, the device is concealed entirely within the nasal passage with no visible external components during use, and it produces a slight, natural change in the appearance of the nose, making it visually discreet. Additionally, because each device acts within the nasal passage, pairs of devices operate independently, and users may apply one device in either nostril or both nostrils depending on their individual needs. Finally, the device is compatible with an intuitive applicator that allows for easy insertion and extraction of the device as well as correct placement (FIGS. 32-35).

In certain embodiments, the disclosure provides a nasal passage dilator for positioning internally within a single nostril, the nostril having a septum, an internal nasal passage, and an interior outer wall surface opposite the septum, the dilator comprising: a first contact plate configured to contact and apply an outward force to the interior outer wall surface when the dilator is positioned in the nasal passage; a second contact plate configured to contact and apply an outward force to the septum when the dilator is positioned in the nasal passage; at least one cross-bridge connecting the first contact plate to the second plate; and a lumen for air flow, wherein the lumen is a hollow space bordered by the first and second contact plates and the at least one cross-bridge, wherein the dilator is configured to rest entirely within the nasal passage of the single nostril.

In exemplary embodiments, the disclosure provides a cylindrical-shaped nasal passage dilator for use within a single nostril, the nostril having an internal nasal passage, a septum, and an interior outer wall surface opposite the septum, the dilator comprising: a first nasal passage contact plate configured to contact and apply an outward force to the septum when the dilator is placed in the nasal passage; a second nasal passage contact plate configured to contact and apply an outward force to the outer wall surface when the dilator is placed in the nasal passage, the first and second contact plates comprising one or more grips to maintain the dilator in place and prevent the dilator from falling out of the nasal passage; two flexible cross-bridges connecting the first contact plate to the second plate, the cross-bridges being oriented such that the first and second contact plates are parallel to a longitudinal axis of the internal nasal passage when the dilator is positioned in the nasal passage; and a slot-shaped lumen for air flow, wherein the lumen is a hollow space bordered by the first and second contact plates and the two cross-bridges, the lumen becoming circular-shaped when the dilator is positioned in the nasal passage, wherein the dilator is configured to rest entirely within the nasal passage of the single nostril.

In other embodiments, the disclosure provides a conical-shaped nasal passage dilator for use within a single nostril, the nostril having an internal nasal passage, a septum, and an interior outer wall surface opposite the septum, the dilator comprising: a first nasal passage contact plate configured to contact and apply an outward force to the septum when the dilator is placed in the nasal passage; a second nasal passage contact plate configured to contact and apply an outward force to the outer wall surface when the dilator is placed in the nasal passage, the first and second contact plates comprising one or more grips to maintain the dilator in place and prevent the dilator from falling out of the nasal passage; two flexible cross-bridges connecting the first contact plate to the second plate, the cross-bridges being oriented such that the first contact plate is parallel to the interior outer wall surface and the second contact plate is parallel to the septum when the dilator is positioned in the nasal passage; and a slot-shaped lumen for air flow, wherein the lumen is a hollow space bordered by the first and second contact plates and the two cross-bridges, the lumen becoming circular-shaped when the dilator is positioned in the nasal passage, wherein the dilator is configured to rest entirely within the nasal passage of the single nostril.

In yet other embodiments, the disclosure provides an A-shaped nasal passage dilator for use within a single nostril, the nostril having an internal nasal passage, a septum, and an interior outer wall surface opposite the septum, the dilator comprising: a first nasal passage contact plate configured to contact and apply an outward force to the septum when the dilator is placed in the nasal passage; a second nasal passage contact plate configured to contact and apply an outward force to the outer wall surface when the dilator is placed in the nasal passage, the first and second contact plates comprising one or more grips to maintain the dilator in place and prevent the dilator from falling out of the nasal passage; a flexible cross-bridge connecting the first contact plate to the second plate, the cross-bridge being oriented such that the first contact plate is parallel to the interior outer wall surface and the second contact plate is parallel to the septum when the dilator is positioned in the nasal passage; and a triangle-shaped lumen for air flow, wherein the lumen is a hollow space bordered by the first and second contact plates and the cross-bridge, the lumen becoming circular-shaped when the dilator is positioned in the nasal passage, wherein the dilator is configured to rest entirely within the nasal passage of the single nostril.

In other embodiments, the disclosure provides a nasal passage dilator for use within a single nostril, the nostril having an internal nasal passage, a nasal bone, a nasal ala, a rim, a bridge, a septum, and an interior outer wall surface opposite the septum, the dilator comprising: a septal contact plate configured to contact and apply an outward force to the septum when the dilator is placed in the nasal passage; a lateral contact plate configured to contact and apply an outward force to the outer wall surface when the dilator is placed in the nasal passage, the tip of the lateral contact plate being configured to align with the nasal bone; a flexible cross-bridge connecting the septal contact plate to the lateral contact plate, the cross-bridge being configured to fit along the bridge of the nose and to bend to provide dilation force; an arm, the arm being configured to fit along a rim of the nostril, and to further fit into one or more pockets in the nasal ala when the dilator is positioned in the nasal passage; and, optionally, one or more removable hooks or external bands, wherein the dilator is configured to rest entirely within the nasal passage of the single nostril.

In some embodiments, the disclosure provides a method of positioning a nasal passage dilator in an internal nasal passage of a single nostril, comprising the steps of: loading the dilator of the disclosure onto an applicator; actuating the applicator to allow the dilator to collapse; inserting the collapsed dilator into the nostril; deploying the dilator in an appropriate position in the internal nasal passage such that the first contact plate contacts and applies an outward force to the septum, the second contact plate contacts and applies an outward force to the interior outer wall surface, and the lumen changes shape to maximize flow of air through the dilator; and retracting the applicator.

In certain embodiments, the disclosure provides a method of dilating a single nostril, comprising the steps of: loading the nasal passage dilator of the disclosure onto an applicator; actuating the applicator to allow the dilator to collapse; inserting the collapsed dilator into the nostril; deploying the dilator in an appropriate position in the internal nasal passage such that the first contact plate contacts and applies an outward force to the septum, the second contact plate contacts and applies an outward force to the interior outer wall surface, and the lumen changes shape to maximize flow of air through the dilator, thereby dilating the nostril; and retracting the applicator.

In other embodiments, the disclosure provides a method of relieving obstruction in a single nostril, comprising the steps of: loading the nasal passage dilator of the disclosure onto an applicator; actuating the applicator to allow the dilator to collapse; inserting the collapsed dilator into the nostril; deploying the dilator in an appropriate position in the internal nasal passage such that the first contact plate contacts and applies an outward force to the septum, the second contact plate contacts and applies an outward force to the interior outer wall surface, and the lumen changes shape to maximize flow of air through the dilator, thereby relieving obstruction in the nostril; and retracting the applicator.

In yet other embodiments, the disclosure provides a method of improving air flow through a single nostril, comprising the steps of: loading the nasal passage dilator of the disclosure onto an applicator; actuating the applicator to allow the dilator to collapse; inserting the collapsed dilator into the nostril; deploying the dilator in an appropriate position in the internal nasal passage such that the first contact plate contacts and applies an outward force to the septum, the second contact plate contacts and applies an outward force to the interior outer wall surface, and the lumen changes shape to maximize flow of air through the dilator, thereby improving air flow through the nostril; and retracting the applicator.

### BRIEF DESCRIPTION OF DRAWINGS

Non-limiting exemplary embodiments are described herein, with reference to the following figures:

FIG. 1 illustrates the problems associated with reconstructive surgery (FIG. 1A) to remove nasal obstruction and the problems associated with nasal dilators currently in use (FIG. 1B).

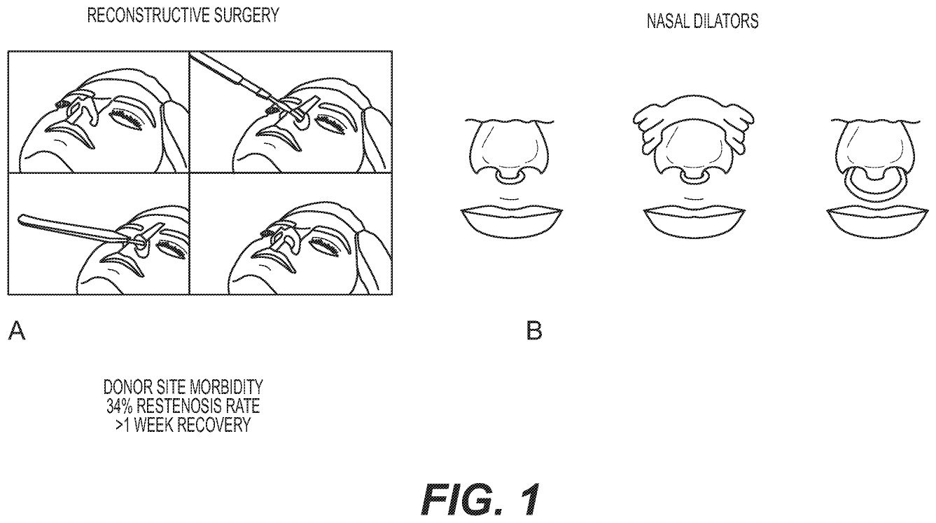

FIG. 2 shows design features and mechanism of action of the present nasal dilator.

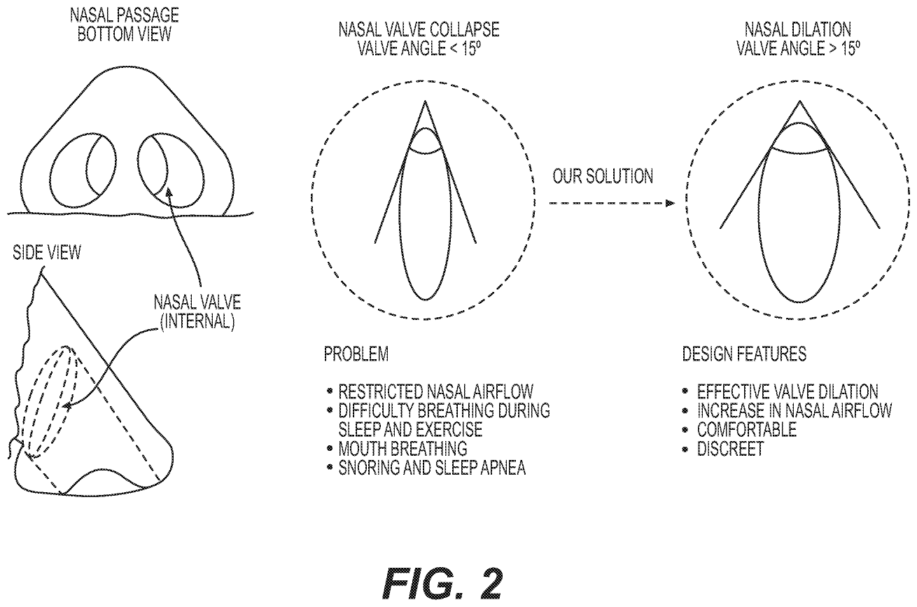

FIG. 3 shows an Internal Skeleton Tension rod dilator. FIG. 3A: shows one view of the Internal Skeleton Tension rod dilator. FIG. 3B: shows another view of the Internal Skeleton Tension rod dilator.

FIG. 4 show one version of a Tapered Tension rod dilator.

FIG. 5 show another version of a Tapered Tension rod dilator with semi-rigid skeleton coated in soft material.

FIG. 6 shows one version of the Triangular wedge dilator.

FIG. 7 shows one view of the Round-padded Tension rod dilator.

FIG. 8 shows another view of the Round-padded Tension rod dilator.

FIG. 9 shows the Tension rod dilator with asymmetrically-elongate pads in dorsal-ventral dimension.

FIG. 10 shows the Tension rod dilator with symmetrically-elongate pads in dorsal-ventral dimension.

FIG. 11 shows one version of the Tension rod dilator with triangular lumen dilator. FIG. 11A depicts one view of the dilator. FIG. 11B depicts another view of the dilator. FIG. 11C depicts the method of inserting the dilator into the nostril of a user. FIG. 11D depicts the correctly positioned dilator in the nasal passage of the user.

FIG. 12 shows one version of the Tension rod dilator with elongate pads in anterior-posterior and ventral-dorsal dimensions.

FIG. 13 shows one version of the Laminar flow feature dilator. FIG. 13A depicts one view of the dilator. FIG. 13B depicts another view of the dilator. FIG. 13C depicts the method of inserting the dilator into the nostril of a user. FIG. 13D depicts the correctly positioned dilator in the nasal passage of the user.

FIG. 14 shows one version of the Elongate-winged dilator. FIG. 14A depicts one embodiment of this version of the dilator. FIG. 14B depicts another embodiment of this version of the dilator. FIG. 14C depicts the correctly positioned dilator in the nasal passage of the user.

FIG. 15 shows one version of the Alignment fin feature with soft surface dilator. FIG. 15A depicts one embodiment of this version of the dilator. FIG. 15B depicts another embodiment of this version of the dilator. FIG. 15C depicts the method of inserting the dilator into the nostril of a user. FIG. 15D depicts the correctly positioned dilator in the nasal passage of the user.

FIG. 16 shows one version of the Semi-tubular dilator.

FIG. 17 shows another version of the Semi-tubular dilator.

FIG. 18 shows yet another version of the Semi-tubular dilator. FIG. 18A depicts the essential features of the dilator. FIG. 18B a removable septum connector attached to the dilator. FIG. 18C depicts the correctly positioned dilator in the nasal passage of a user. FIG. 18D depicts the airflow through the correctly positioned dilator.

FIG. 19 shows one version of the Alignment fin feature with openings dilator. FIG. 19A depicts one embodiment of this version of the dilator. FIG. 19B depicts another embodiment of this version of the dilator. FIG. 19C depicts the method of inserting the dilator into the nostril of a user. FIG. 19D depicts the correctly positioned dilator in the nasal passage of the user.

FIG. 20 shows the notched nose cone dilator.

FIG. 21 shows the inverted notched nose cone dilator.

FIG. 22 shows another view of the Laminar Flow dilator.

FIG. 23 shows one version of the Mechanical Tension Rod dilator.

FIG. 24 shows another version of the Mechanical Tension Rod dilator.

FIG. 25 shows yet another version of the Mechanical Tension Rod dilator.

FIG. 26 shows still another version of the Mechanical Tension Rod dilator. FIG. 26A depicts one view of this version of the dilator. FIG. 26B depicts another view of this version of the dilator.

FIG. 27 shows one version of the Adjustable sizing dilator. FIG. 27A depicts one view of this version of the Watch dilator. FIG. 27B depicts another view of this version of the Watch dilator.

FIG. 28 shows another version of the Adjustable sizing dilator. FIG. 28A depicts one view of this version of the dilator. FIG. 28B depicts another view of this version of the dilator.

FIG. 29 shows the Curved strip dilator. FIG. 29A depicts one view of the dilator. FIG. 29B depicts another view of the dilator.

FIG. 30 shows the Curved strip dilator with septal and lateral pad. FIG. 30A depicts one view of the dilator with septal and lateral pad. FIG. 30B depicts another view of the dilator with septal and lateral pad.

FIG. 31 shows the Ratcheting curved strip dilator (corresponding ratcheting septal pad not shown). FIG. 31A depicts one view of the dilator. FIG. 31B depicts another view of the dilator.

FIG. 32 shows one embodiment of the semi-tubular/modified Triangular wedge dilator. FIG. 32A depicts one view of the semi-tubular/modified Triangular wedge dilator. FIG. 32B depicts the correctly positioned semi-tubular/modified Triangular wedge dilator in the nasal passage of a user. FIG. 32C depicts a modified version of the semi-tubular/modified Triangular wedge dilator correctly positioned in the nasal passage of a user.

FIG. 33 depicts that the semi-tubular/modified Triangular wedge dilator may be connected into full tubes at certain points to aid provide extra mechanical support and potentially provide compatibility with a certain type of applicator tool. FIG. 33A: cross-bridges bend downward. FIG. 33B: cross-bridges bend upward. FIG. 33C: two pairs of cross-bridges bend away from each other to produce a lumen.

FIG. 34 shows that the semi-tubular/modified Triangular wedge dilator may be connected into full tubes at certain points to aid provide extra mechanical support and potentially provide compatibility with a certain type of applicator tool. FIG. 34A: normal semi-tubular/modified Triangular wedge dilator with no cross-bridges. FIG. 34B: semi-tubular/modified Triangular wedge dilator with upturned cross-bridges (parallel to the curvature of the device as a whole). FIG. 34C: semi-tubular/modified Triangular wedge dilator with downturned cross-bridges (essentially turning the semi-tube into a full tube).

FIG. 35 shows the bottom view of the nose with the semi-tubular/modified Triangular wedge dilator and demonstrates the different places a removable connector between dilators or clamps attaching the dilator to the surface of the nose could be positioned.

FIG. 36 shows another embodiment of the semi-tubular/modified Triangular wedge dilator and depicts the method of inserting the dilator into the nostril of a user.

FIG. 37 shows yet another embodiment of the semi-tubular/modified Triangular wedge dilator and depicts the method of inserting the dilator into the nostril of a user.

FIG. 38 depicts applicator tools. FIG. 38A depicts one embodiment of the applicator tool. FIG. 38B depicts another embodiment of the applicator tool.

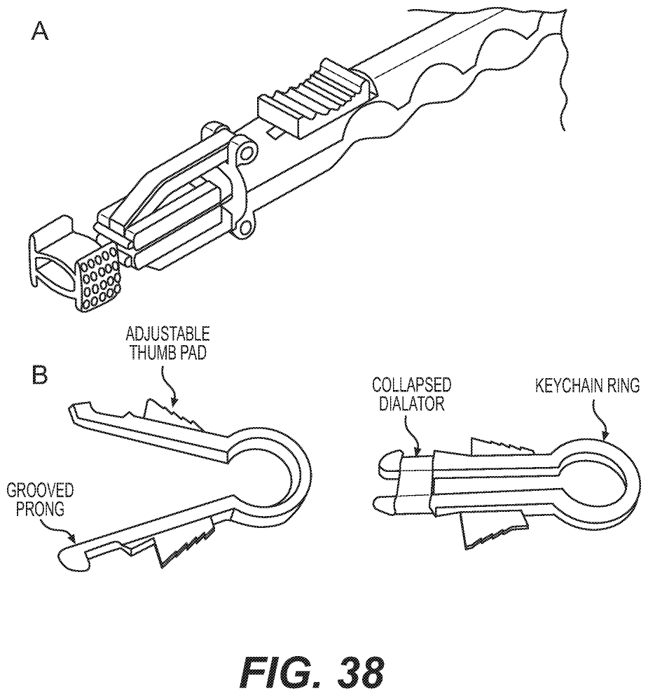

FIG. 39 shows the mechanism by which the prongs of the applicator are operated and mechanically spread apart using an internal mechanism in the applicator.

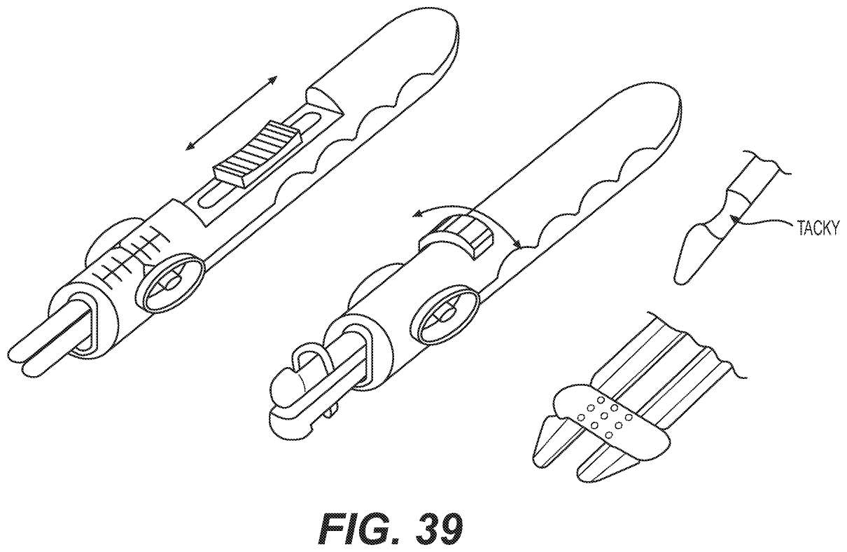

FIG. 40 shows the insertion method with tension rod dilator and applicator. The applicator has groves that provide clearance between the dilator and the nasal passage during insertion so it is shielded and does not rub against the nasal passage and grip prematurely. These grooves also allow the dilator to lock into the applicator so it does not shift during insertion or removal. The angled thumb pads serve as a stopper to guide insertion so the user inserts the dilator on the applicator until the nostril touches the stopper, indicating the dilator is in the correct position. FIG. 41A: dilator and applicator are separate. FIG. 41B: dilator slipped over the prongs of the applicator and into the grooves. FIG. 41C: applicator prongs spring apart and dilator collapses horizontally and expands horizontally to fit beyond nostril and into the nasal passage, which is a vertical oval.

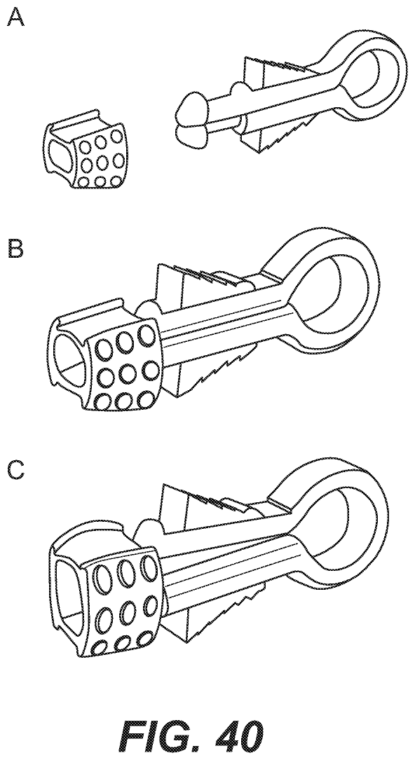

FIG. 41 shows additional insertion tools, using the tension rod dilator as an example. FIG. 41A: the dilator is loaded onto an inner pole that slides in and out of the larger tube by sliding the slider on the hand-piece. The inner pole is ovular in cross section and collapses the dilator horizontally while spreading it vertically, allowing it to fit beyond the nostril and into the nasal passage more easily without touching or gripping the sides. The largest diameter cylinder is a stopper that guides insertion, so the applicator is inserted until the stopper touches the nostrils on the exterior of the nose, indicating the dilator is the optimal distance beyond nostril for deployment. Moving the slider back retracts the inner pole, causing the dilator to retract until it is pressed up against the larger diameter tube. The inner pole retracts fully into the hand-piece, so the dilator is no longer being held in position and immediately deploys. FIG. 41B: the applicator works similarly using a plunger mechanism rather than a slider. Any number of mechanisms could achieve the same effect.

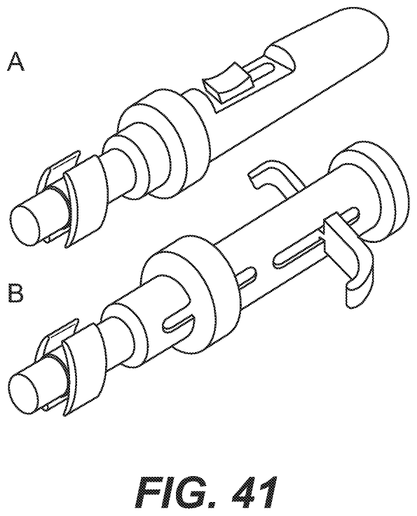

FIG. 42 shows the method for deploying the present device in the internal nasal passage of a single nostril using the applicator.

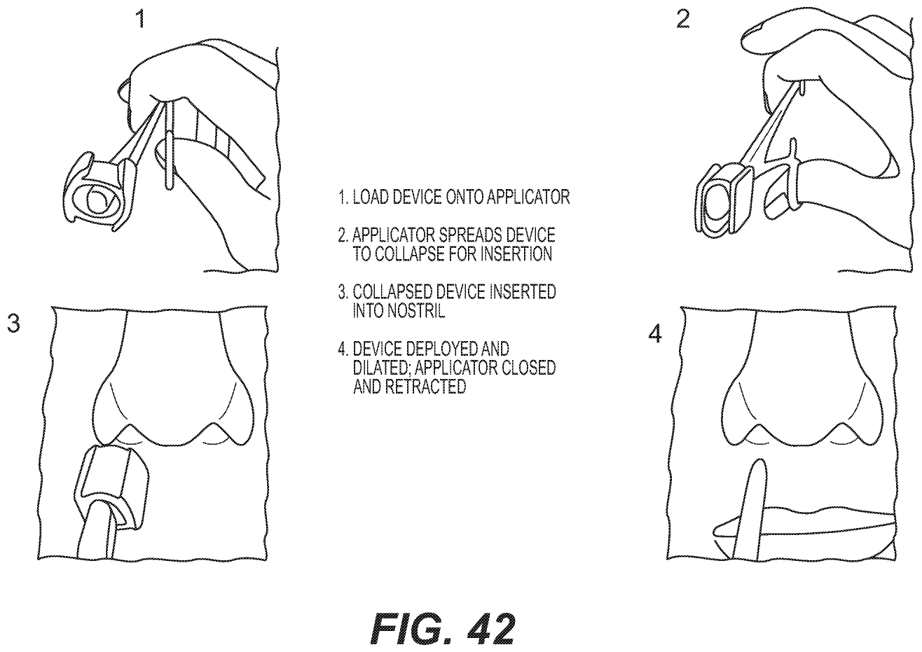

FIG. 43 shows a comparison between a Triangular wedge dilator insertion and a Tapered tension rod dilator insertion.

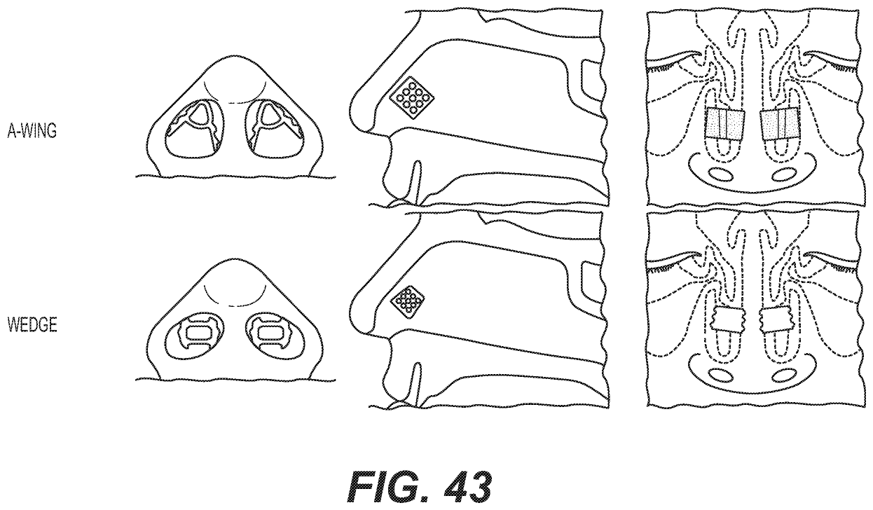

FIG. 44 summarizes the results of an airflow testing study with the present device, and a comparison of the airflow using the present device with that obtained using other available nasal dilators.

### DETAILED DESCRIPTION

For purposes of explanation, specific nomenclature is set forth to provide a thorough understanding of the present disclosure. However, it will be apparent to one skilled in the art that these specific details are not required to practice the aspects of the present disclosure. Descriptions of specific applications are provided only as representative examples. The aspects of the present disclosure are not intended to be limited to the embodiments shown, but are to be accorded the widest possible scope consistent with the principles and features disclosed herein.

The terms “nasal dilation device,” “internal nasal dilator,” “nasal dilator,” “dilator,” “device,” and “present device” are used interchangeably throughout the specification to mean the nasal dilator of the present disclosure. As used herein, the term “actuate” or “actuating” means “to cause a machine or device to operate.”

The present disclosure provides a comfortable and discreet nasal dilation technology with multiple applications across a range of consumer health and clinical settings—daily-time, sleep, and physical activity.

In certain embodiments, the present nasal dilation device is configured to fit most nasal cavities. In specific embodiments, the dilator is configured to fit each nasal cavity independently to dilate each nostril independently and improve airflow. In some embodiments, the dilator is completely internalized within the nose and therefore barely visible or invisible, allowing individuals to use the device during the day without social stigma. Thus, in some embodiments, the nasal passage dilator is configured for positioning internally within a single nostril, the nostril having a septum, an internal nasal passage, and an interior outer wall surface opposite the septum. In specific embodiments, the dilator is configured to rest entirely within the nasal passage of a single nostril. In some embodiments, the device dilates the nostrils to directly counteract nasal valve collapse and increase airflow.

In certain embodiments, the dilator is discreet. In certain aspects, the dilator has no visible external components. In other aspects, the change in aesthetic shape of nose upon deployment of the dilator is unnoticeable due to very slight dilation targeted at the nasal valve. In yet other aspects, the change in aesthetic shape of nose upon deployment of the dilator is natural because dilation occurs evenly along the length of the nose. In some embodiments, the dilator is very comfortable. In certain embodiments, the contact plates of the dilator are made convex in all dimensions and tapered or rounded at the edges for improved comfort and to remove sharp corners and edges that could cause discomfort. In certain aspects, if the contact plates are tapered, they are made to be paper thin so the edges of the pads fold rather than pinch, poke, or dig into the tissue. In other aspects if the contact plates are rounded, the edges are filleted to provide a completely smooth surface so there are no jagged edges.

In certain embodiments, targeting the internal nasal valve directly (as opposed to other areas of the nose) allows device to effectively dilate nasal passage and improve breathing with far less force. In certain aspects, better effects are achieved with less force, producing less discomfort. In some aspects, the dilator exerts slight (for example, about 1N) dilation force directed at key areas on the nasal side wall. In some aspects, the slight dilation force allows device to effectively dilate nasal passage with far less force. In specific embodiments, the dilator pushes out directly against the nasal valve area to relieve nasal obstruction. In other aspects, the shape (curvatures, alignment with nasal anatomy) and material properties of the dilator (soft silicone coating over a relatively rigid skeleton) diffuse pressure for even contact and maximum comfort.

In yet other aspects, the dilator is designed to operate without contacting highly sensitized portions of the nose. In some embodiments, the lack of contact with highly sensitized portions of the nose prevents trigger of sneezes and nasal inflammation. In certain aspects, the dilator has tapered front to air insertion and is designed for collapsibility into a smaller structure so it can be inserted without rubbing against and irritating the internal surface of the nose. In certain embodiments, the dilator is extremely stable. In some aspects, the dilator fits within certain pockets in the nose. The pockets include without limitation the tip, the rim, and the sill of the nose. In specific embodiments, the alignment and stability of the dilator within the nasal passage allows the user to withstand a physiological event such as a sneeze, increased rate of respiration, etc. without the dilator falling out of the nostril. In certain embodiments, the end of the device is aligned with the edge of the nasal bone. In certain aspects, this makes placement of the dilator in the nose more intuitive. In certain embodiments, the portion of the dilator that rests beyond the nostril comprises one or more holes to allow the protrusion of nasal hairs to preserve their natural filtering function. In certain aspects, the one or more holes are present in an alignment feature of the dilator. In other aspects, the one or more holes are present in a stability fit feature of the dilator.

In certain embodiments, the dilator allows for airflow funneling. In certain aspects, the dilator channels air over the middle turbinate, whose mucosa is thought to sense airflow. In certain aspects, When properly aligned, the device has features that direct air across this region to increase the sensation of airflow. In other aspects, the device has features that accelerate air or recruit air. In certain embodiments, the acceleration of air by the dilator involves a narrowing in diameter of the dilator. In some embodiments, the dilator is configured to recruit air by creating a vacuum. In certain aspects, the dilator has one or more fins that allow the device to exert a greater dilation force when air flows past it.

In certain embodiments, the dilator fits in sill beyond nostril and extends into the nasal passage to the point where it is active in the internal nasal valve region. In some embodiments, from side to side, the device is sized to clear fit past the nostril during insertion and provide an effective yet comfortable dilation force without causing excessive or unnatural changes in the appearance of the nose, i.e. by pushing out the nasal sidewall. In other embodiments, vertically, the dilator is sized to fit past the nostril during insertion and span the height of the nasal wall.

In certain embodiments, the dilator can generally be any shape that can be easily inserted in the nostril and does not cause discomfort to the user when inserted within the nostril for an extended time. These shapes include, but are not limited to, cylindrical, triangular, conical, circular, A-shaped, C-shaped, U-shaped, T-shaped, ring-shaped, balloon-shaped, horseshoe shaped, and screw-shaped. In exemplary embodiments, the dilator is cylindrical shaped. In other embodiments, the dilator is conical shaped. In yet other embodiments, the dilator is triangle shaped. In specific embodiments, the dilator is A-shaped.

In particular embodiments, the device is manufactured using soft materials that conform to the geometry of the nasal valve area to diffuse the pressure exerted on the nasal valve. In certain embodiments, the dilator is made of a polymer material. In particular aspects, the dilator is made of a medical grade polymer that is biocompatible and suitable for human use. In specific embodiments, the polymer comprises a biocompatible silicone. In other embodiments, the biocompatible silicone is a tacky, soft silicone. In certain aspects, the tacky, soft silicone is effective in gripping nasal mucosa. In other embodiments, the polymer comprises a plastic. In specific embodiments, the polymer comprises any material that is biocompatible, suitable for human use, and comfortable to the user for extended use.

In certain embodiments, the dilator uses multiple materials, such as a more rigid internal material that provides greater dilation force and coated in a softer external material that is more comfortable against the nasal passage. Therefore, in some embodiments, the dilator comprises a rigid interior framework and a soft exterior surface. In certain embodiments, the rigid interior framework provides greater dilation force. In some embodiments, the rigid interior framework allows the dilator to be correctly positioned inside the nasal passage. In other embodiments, the rigid interior framework prevents the dilator from falling out of the nostril. In yet other embodiments, the rigid interior framework prevents or decreases the dilator from moving within the nostril. In some embodiments, the soft exterior surface causes the dilator to be more comfortably inserted into the nasal passage. In other embodiments, soft exterior surface provides greater adhesion to the dilator positioned in the nasal passage. In certain aspects, the soft exterior allows the dilator to better conform to the nasal passage and diffuse pressure. In other aspects, the soft exterior reduces discomfort to the user associated with rigid objects in the nose for extended periods. In yet other aspects, the soft exterior eliminates the impact of one or more sharp corners and edges of the dilator. In still other aspects, the soft exterior provides tackiness that provides additional grip to the dilator positioned within the nasal passage.

In certain embodiments, the dilator comprises a first contact plate, a second contact plate, at least one cross-bridge connecting the first contact plate to the second plate, and a lumen for airflow. As used herein, the term “lumen” refers to a hollow space bordered by the first and second contact plates and the at least one cross-bridge.

In some embodiments, the first contact plate is configured to contact and apply an outward force to the interior outer wall surface when the dilator is positioned in the nasal passage. In certain embodiments, the second contact plate configured to contact and apply an outward force to the septum when the dilator is positioned in the nasal passage. In some embodiments, the first contact plate and the second contact plate are identical in dimension. In other embodiments, the first contact plate is longer than the second contact plate. In yet other embodiments, the second contact plate is longer than the first contact plate. In some embodiments, the first and second contact plates are straight. In other embodiments, the first and second contact plates are curved. In specific aspects, the curved contact plates are, without limitation, convex, tapered, or rounded. In certain aspects, the curved contact plates allow the dilator to conform to the nasal passage, and therefore improve comfort, stability, usability etc. In specific aspects, the curved first and second contact plates provide added stability to the dilator positioned within the nasal passage.

In particular embodiments, the shape of the device generally conforms to the geometry of the nasal passage, allowing the device to comfortably conform to the existing geometry in its compressed conformation. In specific embodiments, the curvature of the first and second contact plates closely follows the geometry of the nasal valve area when the device is positioned in the nasal passage. In certain embodiments, the alignment of the first and second contact plates matches the shape of the internal nasal passage when the dilator is positioned in the nasal passage. In some embodiments, the first contact plate is parallel to the interior outer wall surface and the second contact plate is parallel to the septum when the dilator is positioned in the nasal passage. In other embodiments, the first and second contact plates are parallel to a longitudinal axis of the internal nasal passage when the dilator is positioned in the nasal passage.

In certain embodiments, the shape of the dilator is customizable to the user's needs. In some aspects, the internal anatomy of the nasal passage is captured in physical models using techniques including, without limitation, physical casts. In other aspects, the internal anatomy of the nasal passage is captured in digital models using techniques including, without limitation, digital scans, MRIs, and CT scans. In certain embodiments, the physical model is converted to a digital model using techniques including, without limitation, three dimensional (3D) scans. In some embodiments, the three dimensional information is into the size, shape, curvatures, and locations of different features of the dilator to produce perfectly customized devices that suit an individual's nose. In certain aspects, the customizable dilators are produced through techniques including, without limitation, injection molding, 3D printing, and polymer casting (similar to the process used to create early prototypes).

In embodiments, the dilator comprises one or more fins. In some embodiments, the one or more fins are configured to exert a dilation force to the nostril when the dilator is in use. In other embodiments, the one or more fins are configured to fit along the rim of the nostril to aid alignment of the device and provide some stability. In certain aspects, the ends of the one or more fins fit into the nasal ala, and the tip of the device fits into the tip of the nose.

In certain embodiments, the nasal dilator comprises one, two, three, four, five, or six cross-bridges. In other embodiments, the nasal dilator has one, two, three, four, five, or six cross-bridges. In exemplary embodiments, the dilator has two cross-bridges. In other exemplary embodiments, the dilator has a single cross-bridge. In certain embodiments, the cross-bridge is rigid. In other embodiments, the cross-bridge is flexible. In yet other embodiments, the dilator comprises of a combination of rigid and flexible cross-bridges. In certain embodiments, the flexible cross-bridge allows the dilator to be easily positioned in the nasal passage. In other embodiments, the cross-bridge bends to provide the force necessary to dilate the nasal passage. In some embodiments, the cross-bridge is curved. In certain embodiments, the curvature in the cross-bridge prevents the dilator from falling out of the nostril. In other embodiments, the curvature in the cross-bridge prevents or decreases movement of the dilator within the nostril. In other embodiments, the dilator does not comprise a cross-bridge.

In certain embodiments, the corners at the attachment points of the first and second contact plates and the cross-bridge are thicker than the rest of the dilator. In some embodiments, the thicker attachment points prevent the dilator from falling out of the nostril. In other embodiments, the thicker attachment points provide greater restoration force when the dilator is positioned in the nasal passage.

In certain embodiments, the lumen facilitates airflow through the nostril when the dilator is positioned in the nasal passage. In some embodiments, the lumen improves airflow through the nostril when the dilator is positioned in the nasal passage. In exemplary embodiments, the lumen maximizes airflow through the nostril when the dilator is positioned in the nasal passage. In other embodiments, the lumen maximizes airflow through the dilator when the dilator is positioned in the nasal passage, thereby relieving nasal obstruction. In some embodiments, the lumen can be any shape that facilitates airflow including, but not limited to slot-shaped, oval-shaped, or triangle shaped. In some embodiments, the lumen is slot-shaped, oval-shaped, or triangle shaped prior to insertion of the dilator in the nostril, but becomes circular-shaped when the dilator is positioned in the nasal passage.

In certain embodiments, the first and second contact plates further comprise a grip. In some embodiments, the grip provides a method for securing the dilator in the nasal cavity during use. In certain aspects, the grip allows the user to secure the dilator themselves with minimal chances of the device falling either into the nasal cavity or out of the nostril during use. In some embodiments, the grip is any structure that prevents the dilator from falling out of the nostril or prevents or decreases movement of the dilator within the nostril. Thus, in certain aspects, the grip prevents the dilator from falling out of the nostril. In other aspects, the grip prevents or decreases movement of the dilator within the nostril, so the dilator remains in its optimal position throughout the duration of use.

In certain embodiments, the grip is an adhesive feature. In certain embodiments, the adhesive feature comprises any geometric pattern including, but not limited to, raised dots, wavy lines, straight lines, gecko adhesion, mounds, stripes, pillars, waves, ridges, spirals, concentric circles, squares, or concentric squares. In exemplary embodiments, the adhesive feature comprises a raised dot adhesive pattern. In other aspects, the device is ribbed to create grip.

In certain embodiments, the adhesive feature comprises one or more suction cups. In certain aspects the adhesive feature comprises a grid of suction cups. In some embodiments, squeezing the nose activates the suction cups and prevents or minimizes movement of the dilator positioned in the nasal passage. In certain embodiments, the adhesive feature comprises one or more mucosal adhesives or other chemical adhesive methods. In certain aspects, the mucosal adhesive activates in mucosal membranes of the nostril and adheres to the nasal wall. In other embodiments, the adhesive feature comprises one or more saline-degradable adhesives. Thus, in certain aspects, saline is sprayed into the nose to degrade the adhesive and extract the dilator. In yet other embodiments, the adhesive feature comprises microvilli that allow for Van der Waal's adhesion.

In certain embodiments, the device uses adhesive means to remain stable within the nasal passage, preventing dislocation caused by sneezing, rapid inspiration, or other physiological factors. In certain embodiments, this allows the device to be safely contained entirely within the nose, precluding the use of visible hooks and clips that existing nasal dilators require for stability and safety.

Certain aspects of the present disclosure thus provide a device comprising two curved contact plates including an adhesive feature. The curvature of the contact plates closely follows the geometry of the nasal valve area when the device is compressed. The adhesive feature comprises any geometric pattern including, but not limited to, raised dots, wavy lines, straight lines, gecko adhesion, mounds, stripes, waves, or pillars. The adhesive feature can also incorporate mucosal adhesives or other chemical adhesive methods. The device further comprises two flexible bands bridging between the curved plates to form an approximately ovular lumen that accommodates airflow.

In other embodiments, the dilator comprises, in addition, one or more attachment means for added stability of the deployed device. In certain embodiments, the attachment means reversibly connect the dilators into a symmetric pair. In other embodiments, the attachment means reversibly connect the individual dilators to the exterior of the nose (whether the columella and septum, the lateral nasal wall, or the crease along the nasal ala). In some embodiments, the attachment means function both to connect the devices to each other and to connect the devices to the exterior of the nose. In certain embodiments, one or more attachment means are configured to be removable. In certain aspects, the one or more attachment means include, without limitation, hooks, clamps, and external bands. In exemplary aspects, the one or more attachment means are hooks. In certain aspects, the hooks, clamps, or external bands are reversibly snapped on and off. In certain aspects, this allows the user to switch the device between “discreet” mode with no visible portions, and “sleep” and/or “athletic” modes wherein the device has externally visible portions during use. In other aspects, this allows the dilator to be worn throughout the day and night, and during exercise.

In certain embodiments, the dilator comprises one or more valves. In certain aspects, the one or valves is a check valve. In some embodiments, the one or more valves are configured to open only when the user is breathing in.

In certain embodiments, in terms of efficacy, by specifically dilating the collapsed internal nasal valve, the present device is able to effectively counteract nasal obstruction at its root cause without exerting excessive forces on the nasal wall. In some embodiments, this design therefore allows the device to be more efficient in its use of dilation force and achieve the same or better results in terms of airflow improvement while exerting significantly less pressure on the nasal valve area.

In some embodiments, the device is tested in order to exert a dilation force that optimizes the dilation force—discomfort relationship. For example, as the nasal valve area is gradually dilated, the airflow through the passage will increase, as will the subject's discomfort. By characterizing this tradeoff between effective dilation and discomfort and designing the device to output the optimal dilation force based on this analysis, the present device is tolerable for extended use during the day while still causing effective dilation.

In certain embodiments, positioning the dilator in the nasal passage causes dilation of the nostril. In other embodiments, positioning the dilator in the nasal passage relieves nasal obstruction. In certain aspects, the nasal obstruction occurs due to narrowing of the internal nasal valve. In other aspects, the nasal obstruction occurs due to collapse of internal nasal valve. In certain embodiments, the nasal obstruction is relieved by widening of internal nasal valve. In some embodiments, the widening of the internal nasal valve is caused by the outward pressure applied by the first and the second contact plates. In certain aspects, positioning the dilator in the nostril causes an increase in nasal valve angle. In some embodiments, the nasal valve angle increases to at least about 15°, at least about 20°, at least about 25°, or at least about 30°. In exemplary embodiments, the nasal valve angle increases to at least about 15°. In specific embodiments, positioning the dilator in the nostril improves flow of air through the nasal passage during inhalaltion. In certain aspects, positioning the dilator in the nostril maximizes flow of air through the nasal passage during inhalation.

In exemplary embodiments, the dilator is cylindrical or conical shaped. In certain embodiments, the dilator has two cross-bridges. In other embodiments, the dilator comprises a rigid interior framework and a soft exterior surface. In certain aspects, the rigid interior framework causes the dilator to better conform to the nasal passage. In other aspects, the rigid interior framework allows the dilator to be correctly positioned inside the nasal passage. In other aspects, rigid interior framework prevents the dilator from falling out of the nostril. In yet other aspects, the rigid interior framework prevents or decreases the dilator from moving within the nostril. In some aspects, the soft exterior surface causes the dilator to be more comfortably inserted into the nasal passage. In particular embodiments, the corners at the attachment points of the first and second contact plates and the cross-bridge are thicker than the rest of the dilator. In certain aspects, the thicker attachment points prevent the dilator from falling out of the nostril. In certain embodiments, the first and second contact plates are parallel to a longitudinal axis of the internal nasal passage when the dilator is positioned in the nasal passage. In some aspects, the lumen is oval-shaped. In some embodiments, the lumen becomes circular when the dilator is positioned in the nasal passage.

In specific embodiments, the dilator is A-shaped. In certain embodiments, the A-shaped dilator has a single cross-bridge. In some embodiments, the top of the A-shaped dilator is curved. In other embodiments, the top of the A-shaped dilator is pointed. In some embodiments, the top of the A-shaped dilator is thicker than the first and second contact plates. In certain aspects, the top of the A-shaped dilator is at least about 0.5 mm, at least about 1.0 mm, at least about 1.5 mm, or at least about 2.0 mm in thickness. In specific embodiments, the top of the A-shaped dilator is at least about 1.5 mm in thickness. In exemplary embodiments, the top of the A-shaped dilator is about 2.0 mm in thickness. In particular embodiments, the thicker top increases the outward force to the septum and the outer wall surface when the dilator is positioned in the nasal passage. In other embodiments, the thicker top prevents the A-shaped dilator from falling out of the nostril. In yet other embodiments, thicker top prevents or decreases movement of the A-shaped dilator in the nostril. In certain embodiments, the first and second contact plates of the A-shaped dilator are of the same length. In other embodiments, the first contact plate is longer than the second contact plate. In certain aspects, the longer first contact plate allows the dilator to be correctly positioned inside the nasal passage. In other aspects, the longer first contact plate prevents or decreases movement of the dilator within the nostril. In some embodiments, the lumen of the A-shaped dilator is triangle or oval-shaped. In certain embodiments, the bottom portion of the contact plates are angled inward to help with insertion of the dilator into the nostril. In certain aspects, the lumen becomes circular shaped when the A-shaped dilator is positioned in the nasal passage.

In certain embodiments, the device is folded into a semi-tube or A-shape that fits along the bridge of the nose in the crease where the nasal septum and sidewall meet. In certain aspects, for stability and more intuitive positioning, the “front” of the device is designed to fit into the pocket at the tip of the nose and has an arm that runs along the rim on the side of the nose just beyond the nostril. In other aspects, the “back” of the device is angled to fit up against the nasal bone. In specific aspects, the design nicely mimics the appearance and functionality of the natural nasal cartilage and directly targets the internal nasal valve.

In certain embodiments, the dilator further comprises one or more filters. In other embodiments, the dilator comprises one or more sensors for airflow. In yet other embodiments, the dilator further comprises one or more chemical sensors. In certain aspects, the chemical sensors include without limitation oxygen, carbon dioxide, and carbon monoxide sensors. In still other embodiments, the dilator further comprises one or more drug delivery mechanisms. In some embodiments, the dilator increases the nasal valve angle using chemical methods. In exemplary embodiments, the dilator contains a ring containing a hydrogel with antihistamine. In some aspects, when the dilator is placed in the nostril, antihistamine is slowly released over time into the nasal airway to help alleviate obstruction. In other embodiments, the device is configured to release essential oils and other compounds associated with aromatherapy.

In certain embodiments, the present device is compatible with an intuitive applicator that spreads the device into a collapsed conformation that is inserted beyond the nasal aperture and into the nasal valve without contacting the walls of the nasal passage. In some embodiments, the applicator then releases dilator, causing it to expand outward and remain in place. In certain aspects, the applicator has features including, without limitation, stoppers and grooves, that guide user to place device in optimal position and at correct angle. In certain aspects, the applicator has features including, without limitation, grooves, notches, and rails, that grip the dilator so it does not deploy from the applicator prematurely. In certain aspects, optimal or correct position of the dilator in the nasal passage is determined by factors including, but not limited to, distance into nasal passage beyond nostril, correct angle, and pitch. In some embodiments, the dilator has tapered front to air insertion and is designed for collapsibility into a smaller structure while still being able to provide the necessary dilation force. In other embodiments, the dilator also has a hidden “pull tab” for quick removal.

In some embodiments, the applicator has a measuring tool and adjustable stopper that indicates to the user the depth to which the device should be inserted within the nose in a particular user to deploy the device directly within the nasal valve area. In certain aspects, once the device is deployed, one side grips onto the lateral nasal wall and the other side grips the nasal septum. In specific aspects, once released from the applicator, the device naturally expands from its collapsed conformation by virtue of the flexibility of its material and geometric properties, and it expands to laterally dilate the nasal valve area and increase airflow.

In some embodiments, the applicator comprises prongs or plates that are mechanically spread apart using an internal mechanism in the applicator. In certain aspects, this mechanism can be activated by a user to manipulate the prongs through the use of a slider, button, or roller. In certain embodiments, the applicator also comprises a feature that retracts the prongs to different distances, as through a ratcheting mechanism or some other mechanism in order to tune the distance the prongs reach into the nasal passage beyond the aperture, thereby determining the placement of the device. In specific embodiments, the applicator is tunable to vary deployment location. In certain embodiments, the applicator comprises ratchet lateral sliders to extend prongs. In some embodiments, the dorsal slider is moved with a thumb to spread the prongs of the applicator. In certain aspects, the prongs have divots to secure and shield the dilator until deployment.

In some embodiments, the applicator also comprises a measuring tool that allows users to determine the location of their nasal valve and then set the applicator to retract the prongs to the appropriate location so that the device is deployed at precisely the right distance into the nasal valve. In certain embodiments, the specific mechanisms for spreading the prongs and retracting the prongs include any combination of simple machines including, but not limited to, gear systems, screws, and wedges.

In specific embodiments, the applicator is intuitive to use to insert the device, extract the device, and adjust the device as necessary throughout the day. In certain embodiments, since the dilator is not coupled together as a symmetric pair, users can insert devices into single nostrils independently. For example, users can insert dilators into only the right nostril, only the left nostril, or both nostrils depending on their needs. In some embodiments, by virtue of the flexibility of the dilator and its natural tendency to expand once deployed in the valve area, the device expands to dilate nasal valve areas of different sizes. This allows the dilator to be highly versatile and used effectively in nasal valve areas of different sizes and geometries.

In certain embodiments, the applicator allows for a high degree of usability. In some embodiments, the pen design, plunger design, or tweezers design of the applicator considers human factors. In certain aspects, the applicator is used to both deploy and extract the dilator. In certain embodiments, the applicator ensures that the dilator is deployed in the correct location and position within the nasal passage. In certain aspects, the applicator measures the diameter of the nostril to determine the correct location and position to deploy the dilator.

Another aspect of the present disclosure provides a method for deploying the device in a nasal passage. The method comprises loading the device onto the prongs of the applicator; spreading apart the prongs of the applicator, thereby spreading the flexible bands on the device apart from each other, causing the elastic material to stretch outward and pull the adhesive plates inward so that the device is in a collapsed conformation that can fit within the nasal aperture and within the nasal valve area; inserting the device into the nose on the applicator prongs, moving the applicator into the nose until the ends of the prongs reach the nasal aperture, at which point the applicator will not be able to move further into the nose, thereby indicating that the contracted device is positioned within the nasal valve correctly. The method further comprises releasing the device from the applicator and allowing the adhesive plates to elastically expand outward to make contact with and dilate the nasal valve. The device will expand until the dilation force imposed by the device matches the normal force imposed by the nasal passage. At this point, the device is in a partially compressed conformation and the internal lumen for airflow is approximately circular.

In certain embodiments, the disclosure provides a method of positioning a nasal passage dilator in an internal nasal passage of a single nostril, comprising the steps of: loading the dilator of the disclosure onto an applicator; actuating the applicator to allow the dilator to collapse; inserting the collapsed dilator into the nostril; deploying the dilator in an appropriate position in the internal nasal passage such that the first contact plate contacts and applies an outward force to the septum, the second contact plate contacts and applies an outward force to the interior outer wall surface, and the lumen changes shape to maximize flow of air through the dilator; and retracting the closed applicator.

In some embodiments, the present device is useful as a breathing aid for daily use. In other embodiments, the present device is useful as a sleep aid for the reduction or prevention of snoring or in the treatment of sleep disorders including, but not limited to, sleep apnea. In some embodiments, the present device is useful as an aid in athletics or exercise to enhance/improve breathing of the subject. In some embodiments, the device is useful in all scenarios, including, without limitation, use throughout the day, at night, and during exercise.

In some embodiments, the present device is useful as an intranasal or intraluminal drug delivery platform. In some further embodiments, the drug is embedded or absorbed into the device. In other further embodiments, a drug may be applied to an internal or external surface of the device as a liquid, gel or powder. In some embodiments of a drug delivery platform, delivery of the drug is by absorption into tissues contacting the device. In other embodiments of a drug delivery platform, delivery of the drug is by insufflation of the drug off the device and into the airway of the subject.

In some embodiments, the present device is useful in a wearable sensor. In some further embodiments, the sensor detects airflow, respiration rate, humidity, or airborne particulates, pathogens or organisms. In some embodiments, the present device is useful as a stent or support in other bodily lumens including, but not limited to, the trachea or blood vessels. In some embodiments, the present device is useful as a surgical retractor, or port, to expand an incision or other entry portal for the introduction of surgical instruments or provide operating space.

In certain embodiments, the disclosure also provides a mold for casting the present dilators. In some embodiments, the mold is a one-part mold. In certain aspects, the one-part mold comprises an overflow chamber to allow the entire device to be captured by the material. In specific embodiments, the one-part mold comprises a removable base. In other embodiments, the mold is a two-part mold. In certain aspects, the two-part mold is 3D-printed. In other aspects, the mold is cast metal or silicone. In other aspects, the two-part mold allows for easy removal and quick casting of the device in a variety of materials. In exemplary aspects, the two-part mold comprises elevated pillars to facilitate removal of the device after casting.

The disclosure further provides a method for polymer casting. In exemplary embodiments, the method for polymer casting comprises: spraying mold release onto the assembled mold, poring equal parts of silicone-base and a cross-linker into a dish or a cup, mixing the components thoroughly, and injecting polymer into the mold. In other embodiments, a method for post-processing the polymer comprises: allowing the polymer to cure in the mold, removing base and pins from the mold, removing the polymer from the mold, cutting outline of dilator from excess polymer, sliding device on a pin to slice away the excess material, and separating pin and excess material to obtain the finished dilator.

### Exemplary Embodiments

The following paragraphs provide several exemplary embodiments of the present dilator. In general, the dilator provides a nasal breathing aid suitable for use throughout the day. In certain aspects, much like a contact lens, users insert and remove the device daily, experience an instant improvement in nasal breathing upon insertion, and comfortably wear the device in public without detection. Rather than undergo invasive nasal reconstruction, users will deploy the dilator to breathe better throughout the day, at night, and during exercise.

In certain embodiments, the device is a tapered silicone stent consisting of two flexible cross-bridges bridging two soft contact plates whose shape closely follows the complex internal nasal anatomy. When deployed, one contact plate grips the nasal septum and the other presses against the lateral nasal wall to dilate the passage and stent it open. This dilation force comes from the two flexible cross-bridges, which bend to provide a gentle spring force while forming a lumen to accommodate airflow.

In specific embodiments, to insert the dilator, the user squeezes a compatible applicator tool and slides the dilator over the prongs. When released, the prongs naturally spring apart to stretch the dilator vertically and collapse it horizontally so it can fit beyond the nostril and into the nasal vestibule. The user then inserts the prongs until the nostril meets the thumb pads, indicating the dilator is positioned optimally within the nose. Squeezing the applicator deploys the dilator, which immediately expands in place and stents the nasal passage. Importantly, the dilator can be used without the applicator, which is designed to facilitate rather than limit usability.

In certain aspects, the dilator has the potential to improve of the quality of life for the millions of nasal obstruction sufferers who currently forgo treatment. Faced with the choice between inaction and the appointments, extensive recovery, and uncertain outcome inherent with surgery, the majority of individuals—especially those with minor to moderate obstruction—elect to live with their symptoms, which present a constant bother, source of embarrassment, and long-term health risk. However, these same individuals insist the present device presents a vastly preferred solution to aid their breathing during the day.

In certain embodiments, the dilator's key value lies in its ability to instantly enhance nasal breathing without the risk and inconvenience of reconstructive surgery or the stigma and discomfort of prevailing sleep aid nasal dilators. Existing sleep aids use visible clamps to secure the device in place and enable correct insertion and quick removal. However, these features are so embarrassing that sufferers will use the devices at home overnight but remove them before leaving for work or school. In contrast, to present dilator's fit within the nasal passage and its mechanical and material properties allow it to remain stable in the nose without external attachments, even during a sneeze.

Moreover, the applicator allows users to very easily and accurately insert, remove, and adjust the device within the nose, rendering external components unnecessary for usability. With minimal practice, users successfully insert the device in seconds. The dilator's ease of use and low requirements for maintenance allow it to easily integrate into users' daily routines.

In specific embodiments, for comfort, the dilator is coated in soft silicone that conforms to the nasal passage to distribute pressure evenly. Moreover, by specifically targeting the internal nasal valve—the flow limiting segment within the nose—the device is able to effectively counteract nasal obstruction at its source, achieving superior results with significantly less pressure than existing products. These features make the present device comfortable enough to wear continuously for an entire day.

Internal Skeleton Tension Rod Design

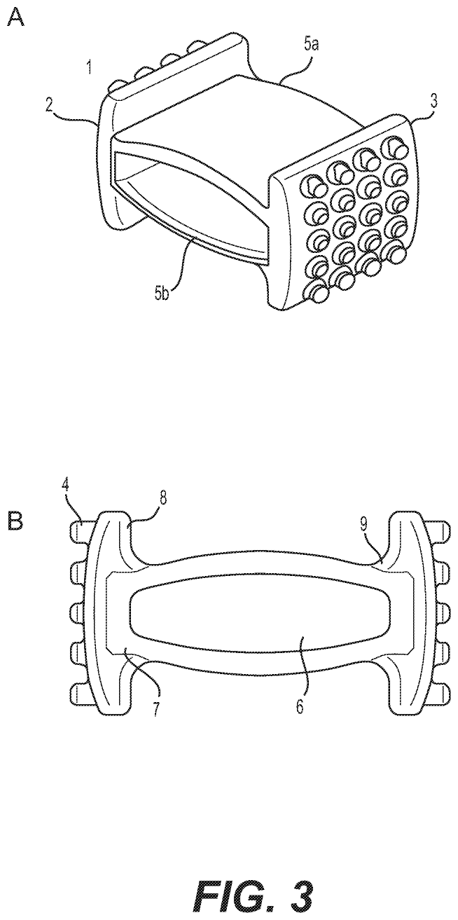

FIGS. 3A and 3B show an Internal Skeleton Tension rod dilator (1). The dilator is a cylindrical-shaped nasal passage dilator for use within a single nostril, comprising: a first nasal passage contact plate (2) configured to contact and apply an outward force to the septum when the dilator is placed in the nasal passage; a second nasal passage contact plate (3) configured to contact and apply an outward force to the outer wall surface when the dilator is placed in the nasal passage, the first and second contact plates comprising a raised dot adhesive pattern (4) to maintain the dilator in place and prevent the dilator from falling out of the nasal passage; two flexible cross-bridges (5a and 5b) connecting the first contact plate to the second plate, the cross-bridges being oriented such that the first and second contact plates are parallel to a longitudinal axis of the internal nasal passage when the dilator is positioned in the nasal passage; and a slot-shaped lumen (6) for air flow, wherein the lumen is a hollow space bordered by the first and second contact plates and the two cross-bridges, the lumen becoming circular-shaped when the dilator is positioned in the nasal passage, wherein the dilator is configured to rest entirely within the nasal passage of the single nostril.

The dilator further comprises a rigid interior framework (7) and a soft, tacky exterior surface or coating (8). The flexible cross-bridges can be easily compressed by pressing the contact plates inward. The rigid interior framework utilizes the restoring force from the deformation of the device (by pressing the contact plates inward) to supply a force to the specific point of the nostril. The soft exterior surface causes the dilator to be more comfortably inserted into the nasal passage and provides greater adhesion. Further, the corners at the attachment point of the contact plates (9) to the cross-bridges are thicker, which prevent the dilator from falling out of the nostril. The first and second contact plates of the dilator provide large areas to the nostril and septum. Additionally, the contact plates provide a raised dot adhesive pattern to prevent the device from slipping or falling out of the nostril when in use.

This design better conforms to the nasal passage. The dilator can be deformed to fit in many different sized or shaped nasal passages. In addition, the dilator provides a robust design that can be easily inserted and extracted with an applicator.

The Tapered Tension Rod Design

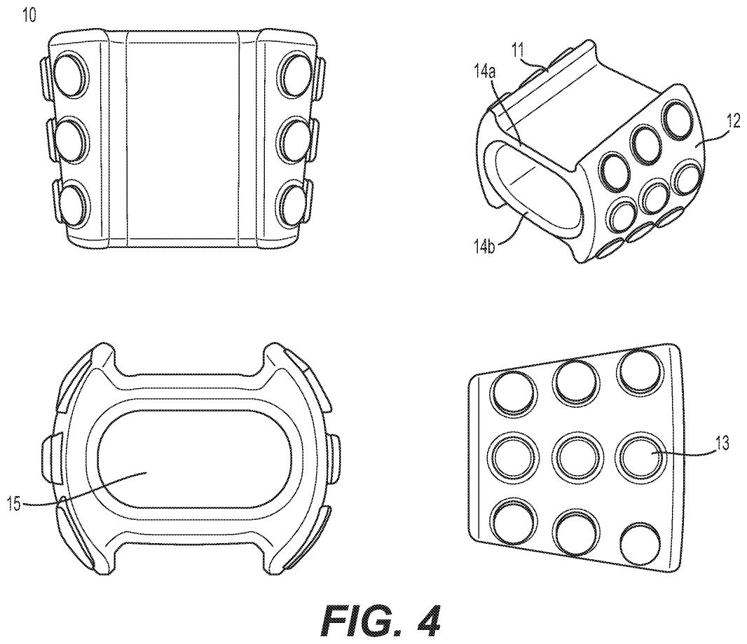

FIG. 4 show one version of the Tapered Tension rod dilator (10). The dilator is a conical-shaped nasal passage dilator for use within a single nostril comprising: a first nasal passage contact plate (11) configured to contact and apply an outward force to the septum when the dilator is placed in the nasal passage; a second nasal passage contact plate (12) configured to contact and apply an outward force to the outer wall surface when the dilator is placed in the nasal passage, the first and second contact plates comprising a raised dot adhesive pattern (13) to maintain the dilator in place and prevent the dilator from falling out of the nasal passage; two flexible cross-bridges (14a and 14b)connecting the first contact plate to the second plate, the cross-bridges being oriented such that the first contact plate is parallel to the interior outer wall surface and the second contact plate is parallel to the septum when the dilator is positioned in the nasal passage; and a slot-shaped lumen (15) for air flow, wherein the lumen is a hollow space bordered by the first and second contact plates and the two cross-bridges, the lumen becoming circular-shaped when the dilator is positioned in the nasal passage, wherein the dilator is configured to rest entirely within the nasal passage of the single nostril.

This design provides a good fit in the nasal passage and facilitates insertion into the nasal passage. The design further prevents slippage of the inserted device in the nasal passage. The conical shape of the dilator mirrors the nasal anatomy and allows the dilator to fit more naturally in the nasal passage. The slot-shaped lumen becomes circular in the nasal passage to increase laminar flow.

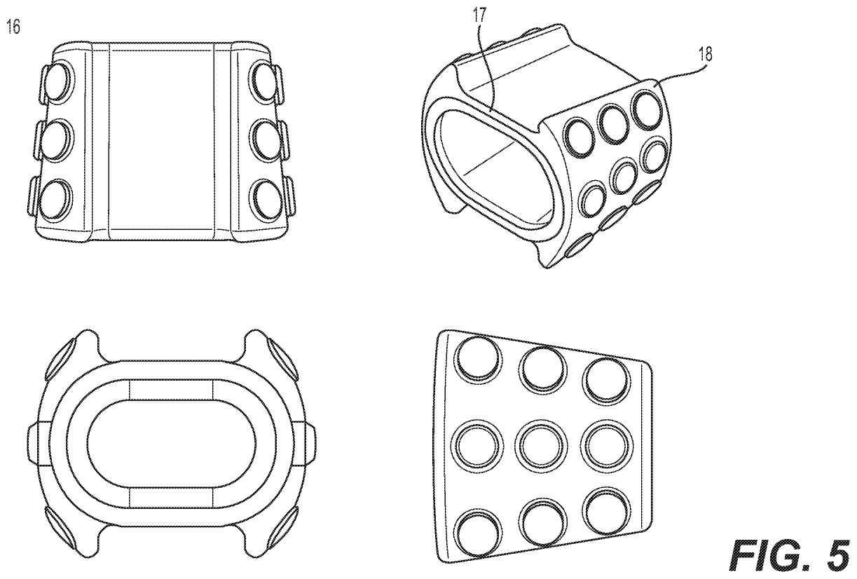

FIG. 5 show another version of the Tapered Tension rod dilator with semi-rigid skeleton coated in soft material (16). In addition to the version depicted in FIG. 4, this dilator comprises a rigid interior framework (17) and a soft, tacky exterior surface or coating (18). The flexible cross-bridges can be easily compressed by pressing the contact plates inward. The rigid interior framework utilizes the restoring force from the deformation of the device (by pressing the contact plates inward) to supply a force to the specific point of the nostril. The soft exterior surface causes the dilator to be more comfortably inserted into the nasal passage and provides greater adhesion. Further, the corners at the attachment point of the contact plates to the cross-bridges are thicker, which prevent the dilator from falling out of the nostril. The first and second contact plates of the dilator provide large areas to the nostril and septum. Additionally, the contact plates provide a raised dot adhesive pattern to prevent the device from slipping or falling out of the nostril when in use. This design better conforms to the nasal passage. The dilator can be deformed to fit in many different sized or shaped nasal passages. In addition, the dilator provides a robust design that can be easily inserted and extracted with an applicator.

The Triangular Wedge Design

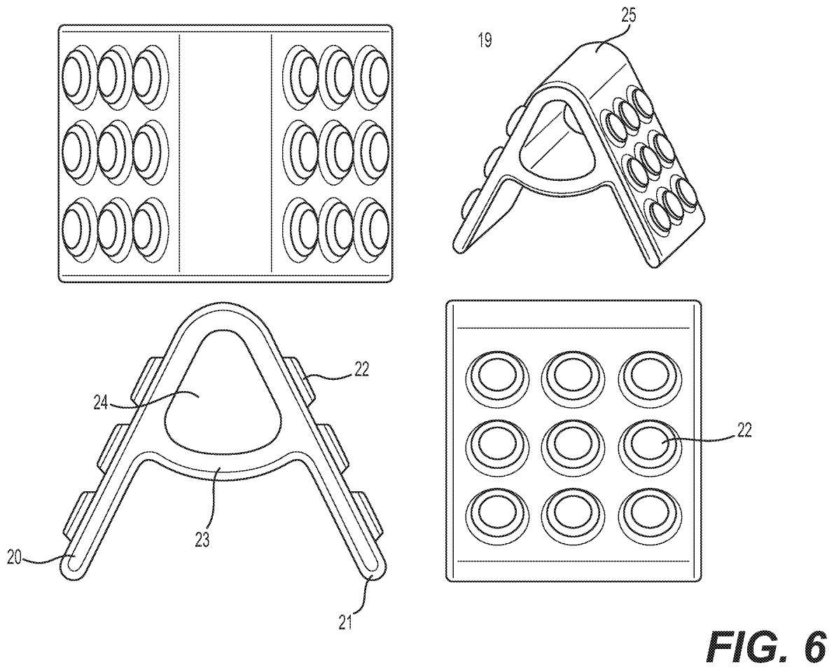

FIG. 6 shows a Triangular Wedge dilator (19). The dilator is an A-shaped nasal passage dilator for use within a single nostril, the dilator comprising: a first nasal passage contact plate (20) configured to contact and apply an outward force to the septum when the dilator is placed in the nasal passage; a second nasal passage contact plate (21) configured to contact and apply an outward force to the outer wall surface when the dilator is placed in the nasal passage, the first and second contact plates comprising a raised dot adhesive pattern (22) to maintain the dilator in place and prevent the dilator from falling out of the nasal passage; a flexible cross-bridge (23) connecting the first contact plate to the second plate, the cross-bridge being oriented such that the first contact plate is parallel to the interior outer wall surface and the second contact plate is parallel to the septum when the dilator is positioned in the nasal passage; and a triangle-shaped lumen (24) for air flow, wherein the lumen is a hollow space bordered by the first and second contact plates and the cross-bridge, the lumen becoming circular-shaped when the dilator is positioned in the nasal passage, wherein the dilator is configured to rest entirely within the nasal passage of the single nostril.

The Triangular Wedge dilator has a single cross-bridge (23). The increased curvature of the cross-bridge prevents slippage and results in a taller overall device. The top (25) of the Triangular Wedge d dilator is curved. In one aspect, the top of the Triangular Wedge dilator is about 1.5 mm in thickness. In another aspect, the top of the Triangular Wedge dilator is about 2.0 mm in thickness. The design increases the outward restoring force to the septum and the outer wall surface when the Triangular Wedge dilator is positioned in the nasal passage. Additionally, the design prevents the Triangular Wedge dilator from falling out of the nostril. The design also prevents or decreases movement of the Triangular Wedge dilator in the nostril. The lumen becomes circular shaped when the Triangular Wedge dilator is positioned in the nasal passage.

This design results in less material along the bottom of the nasal passage. The triangular shape better approximates the geometry of the nasal passage, and the height of the device prevents rotation within the nasal passage. The design minimizes the slippage and spinning of the device in the nasal passage by making the contact plates too long to fit sideways in the nasal passage. The airflow also goes beneath the device to fit better with natural airflow. Further, the design minimizes the slipping that takes place during squeezing to restrict the device to generating only symmetric outer force. Additionally, the “A” shape fits more naturally in the nasal passage and provides a better fit to the curvature of the top of the nasal passage. The design is additionally advantageous in that it is extremely discreet.

This design is inspired by the natural nasal cartilage. Essentially, when positioned in the nose, the device supplements the alar and lateral cartilage that is already there by pushing off the septum onto the lateral wall. For stability, this concept also includes a fin that runs along the rim of the nostril (hidden by the slight sill the nostril creates) and fits into the space at the nasal tip and wings. The contact plates on this device are made convex and tapered or rounded at the edges for improved comfort and to remove “sharp” corners and edges that could cause discomfort. The lateral contact plate is designed to push out in the same areas where functional rhinoplasty grafts are often place and where the modified cattle maneuver is performed to test the nasal valve area. The distal portion of this contact plate (further into the nose) is designed to align with the nasal bone.

Key difference from Round-padded Tension rod dilator to Triangular Wedge dilator is that the lateral and septum contact plates are connecting on top. This wedge shape fits into the groove along the bridge of the nose between the lateral wall and the septum. This design better accommodates the “triangularity” of the nose—the Round-padded Tension rod dilator designs tend to rotate or buckle because the nose is not a straight tube—the lateral wall is at an angle to the septum. Having the contact plates connected at the top and pushing out at an angle overcomes this problem and increases stability and effectiveness.

In another design, the device is folded into an A-shape that fits along the bridge of the nose in the crease where the nasal septum and sidewall meet. For stability and more intuitive positioning, the “front” of the device is designed to fit into the pocket at the tip of the nose and has an arm that runs along the rim on the side of the nose just beyond the nostril. The “back” of the device is angled to fit up against the nasal bone. The design nicely mimics the appearance and functionality of the natural nasal cartilage and directly targets the internal nasal valve.

The Round-Padded Tension Rod Design

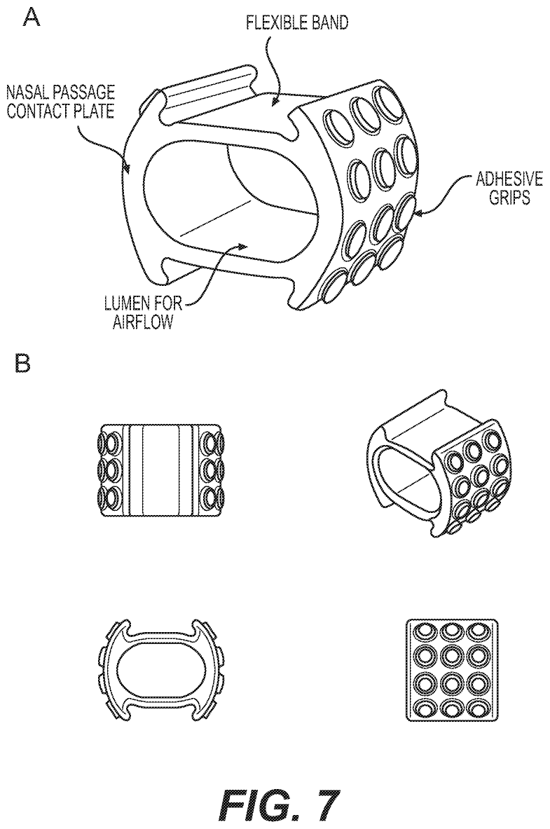

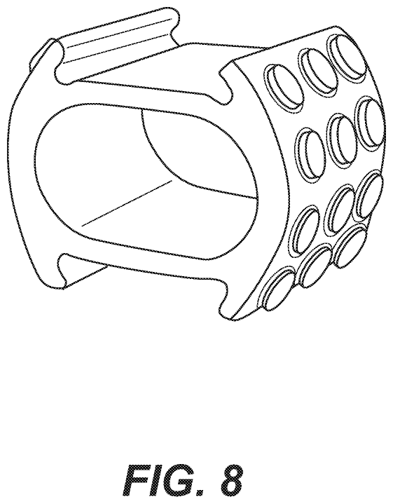

FIGS. 7A, 7B, and 8 depict the Round-padded Tension rod dilator. The dilator has curved first and second contact plates, two flexible cross-bridges, and a slot-shaped lumen. The first and second contact plates of the dilator additionally contain a raised dot adhesive pattern to prevent the device from slipping or falling out of the nostril when in use. The adhesive pads conform to the nasal passage and grip for stability. The flexible cross-bridges allow the device to be compressed in order to be inserted in the nostril. The spring force in the compressed cross-bridges dilates the nasal valve when the device is in use. The dilator is made with biocompatible silicone and is engineered to balance dilation force with comfort. When in use, the device rests entirely within the nasal passage and causes limited aesthetic deformation of the nose.

This design increases adhesion by having the first and second contact plates better conform to the shape of the nasal passage due to their curved shape. Additionally, the slot-shaped lumen allows for maximized airflow because the lumen changes shape when the device is positioned in the nasal passage. In certain aspects, the lumen becomes circular when the device is positioned in the nasal passage.

In essence, the device is a tapered silicone stent consisting of two flexible cross-bridges bridging two soft contact plates whose shape closely follows the complex internal nasal anatomy. When deployed, one contact plate grips the nasal septum and the other presses against the lateral nasal wall to dilate the passage and stent it open. This dilation force comes from the two flexible cross-bridges, which bend to provide a gentle spring force while forming a lumen to accommodate airflow.

To insert the device, the user squeezes a compatible applicator tool and slides the dilator over the prongs. When released, the prongs naturally spring apart to stretch the dilator vertically and collapse it horizontally so it can fit beyond the nostril and into the nasal vestibule. The user then inserts the prongs until the nostril meets the thumb pads, indicating the dilator is positioned optimally within the nose. Squeezing the applicator deploys the dilator, which immediately expands in place and stents the nasal passage. Importantly, the device can be used without the applicator, which is designed to facilitate rather than limit usability.

The Tension Rod Dilator with Asymmetrically-Elongate Pads in Dorsal-Ventral Dimension Design

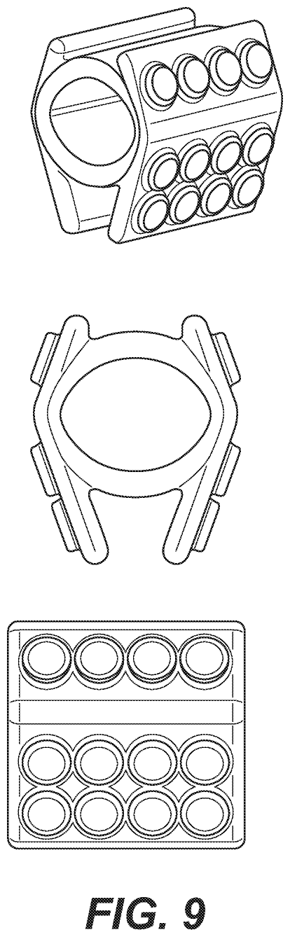

FIG. 9 shows the Tension rod dilator with asymmetrically-elongate pads in dorsal-ventral dimension. The dilator has bent first and second contact plates, wherein the top portion of the contact plates is shorter than the bottom portion of the contact plates. The dilator further has two curved, flexible cross-bridges and an oval-shaped lumen. The shorter top contact plates allow the device to more closely follow the nasal anatomy and fit more naturally in the nasal passage. The first and second contact plates additionally contain a raised dot adhesive pattern to prevent the device from slipping or falling out of the nostril when in use.

This design f reduces slippage and increases the outward force at the front of the nasal valve.

The Tension Rod Dilator with Symmetrically Elongate Pads in Dorsal-Ventral Dimension Design

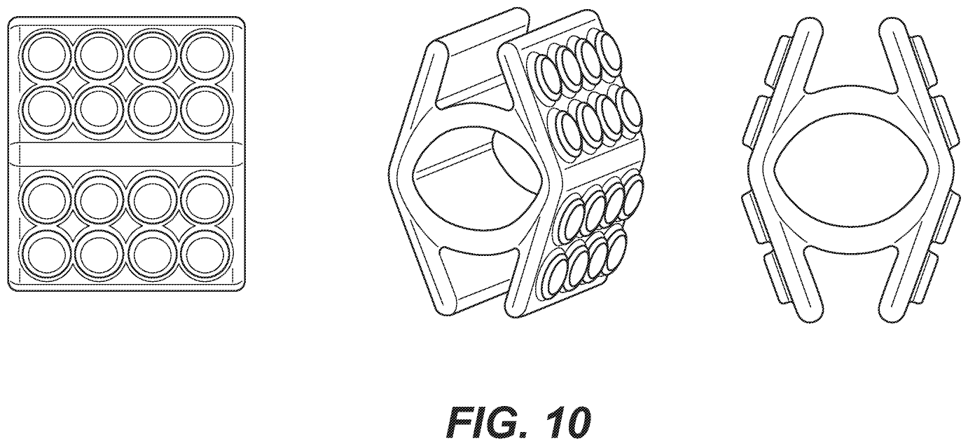

FIG. 10 shows the Tension rod dilator with symmetrically-elongate pads in dorsal-ventral dimension. The dilator has bent first and second contact plates, wherein the top and bottom portions of the contact plates are of the same length. The dilator further has two curved, flexible cross-bridges and an oval-shaped lumen. The taller contact plates prevent slippage and the taller top contact plates exert a force on the front of the nasal valve. The first and second contact plates additionally contain a raised dot adhesive pattern to prevent the device from slipping or falling out of the nostril when in use.

The device is a tapered silicone stent consisting of two flexible cross-bridges bridging two soft contact plates whose shape closely follows the complex internal nasal anatomy. When deployed, one contact plate grips the nasal septum and the other presses against the lateral nasal wall to dilate the passage and stem it open. This dilation force comes from the two flexible cross-bridges, which bend to provide a gentle spring force while forming a lumen to accommodate airflow. The contact plates are large to diffuse force over the septum and cartilage. Rounded edges prevent uncomfortable point load. The cross-bridges are filleted and pre-bent to provide directionality during compression to prevent buckling. The contact plates potentially have a grip pattern to increase surface area contact with nasal passage.

The dilator has dual material: relatively stiff skeleton over-molded with soft gel skin. Insert provides necessary dilation force while coating distributes pressure evenly and conforms to nasal passage slightly to enhance comfort and stability. Natural aesthetic change in the appearance of the nose—exerts the minimum dilation force necessary to effectively relieve symptoms. Targets dilation at the internal nasal valve to achieve this effect. Device runs along the length of the nose to provide dilation evenly so it looks natural and less noticeable (as opposed to applying pressure at discreet points and creating a noticeable tent-like effect. The device contains a reversibly removable septum ring or removal external clip so the user can switch freely between a “discreet mode” where the device is safely worn without any external components and wearing the device at night or during exercise with the extra stability a clip would provide). The design avoids parts of the nose that are sensitive and trigger sneeze. The dilator also provides a mechanism of accelerating air over space between middle and inferior turbinate by redirecting (aiming air at middle space), accelerating (funneling air to increase velocity) and channeling (producing laminar flow in a protected straight tube) air to increase sensation of nasal breathing. The dilator also contains a component that leads to vacuum effect.

The dilator is compatible with an intuitive applicator tool that collapses the dilator for easy insertion and accurate placement within the nasal passage. The applicator guides users to the internal nasal valve—the flow-limiting segment within the nose—to directly counteract nasal obstruction at its source. The guidance is achieved by adjustable stoppers or adjustable prongs on the applicator tool the user can adjust based on nasal measurements. The same application mechanism worts in reverse for safe and fast removal. The device also contains a quick pull tab a fast release mechanism so device can be removed without applicator. Tab is hidden in tip or side of nose (doubles as stability fin in most embodiments).

This design fits more naturally in the nasal passage and therefore reduces slippage and increases the outward force at the front of the nasal valve.

The Tension Rod Dilator with Triangular Lumen Design

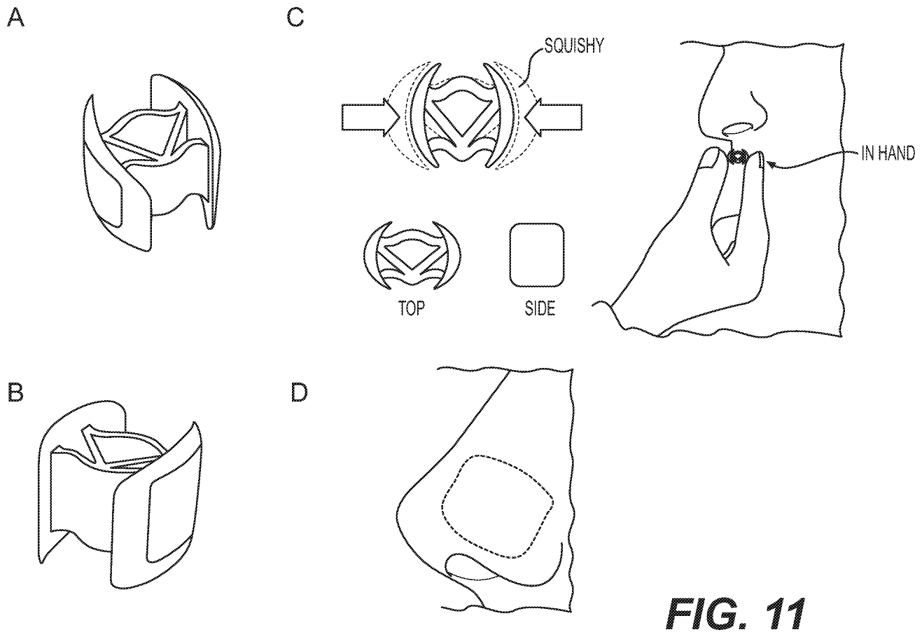

FIG. 11 shows one version of the Tension rod dilator with triangular lumen. FIG. 11A depicts one view of the dilator. FIG. 11B depicts another view of the dilator. The dilator contains two identical, flexible, curved contact plates connected by two cross-bridges. The cross-bridges are further supported by a rigid inner triangular structure creating a triangular lumen. The triangular structure provides the strongest shape and provides added support. The contact plates are squeezed during insertion to allow placement in the nasal airway. FIG. 11C depicts the method of inserting the dilator into the nostril of a user. When in use, the outward pressure from the supported contact plates relieves nasal obstruction. FIG. 11D depicts the correctly positioned dilator in the nasal passage of the user.

The Tension Rod Dilator with Elongate Pads in Anterior-Posterior and Ventral-Dorsal Dimension Design

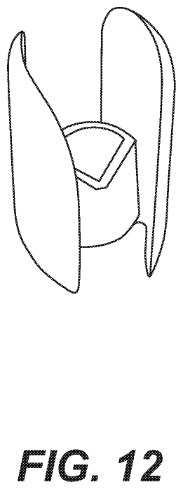

FIG. 12 shows another version of the Tension rod dilator with elongate pads in anterior-posterior and ventral-dorsal dimension. The dilator contains two identical, flexible, curved contact plates connected by two cross-bridges. The cross-bridges are further supported by a rigid inner triangular structure creating a triangular lumen. The triangular structure provides the strongest shape and provides added support. When in use, the outward pressure from the supported contact plates relieves nasal obstruction. The contact plates in this design are longer to diffuse the pressure on the septum and the nostril allowing for a more comfortable wear.

The Laminar Flow Feature Design 1

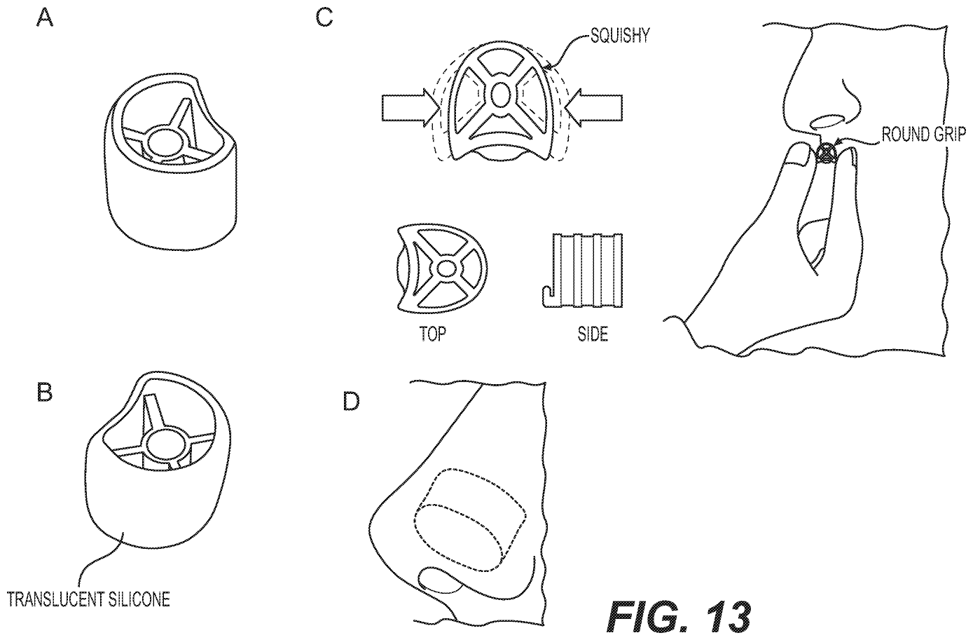

FIG. 13 shows one version of the Laminar flow feature dilator. The dilator is made of translucent silicone and contains a flexible, outer semi-circular surface attached to a circular lumen via multiple braces. The protected lumen in this device is a straight tube to produce laminar flow and channel air over the portion of the nasal cavity that senses airflow. FIG. 13A depicts one view of the dilator. FIG. 13B depicts another view of the dilator. FIG. 13C depicts the method of inserting the dilator into the nostril of a user. FIG. 13D depicts the correctly positioned dilator in the nasal passage of the user. When in use, the outward pressure from the outer surface relieves nasal obstruction.

The Elongate-Winged Design

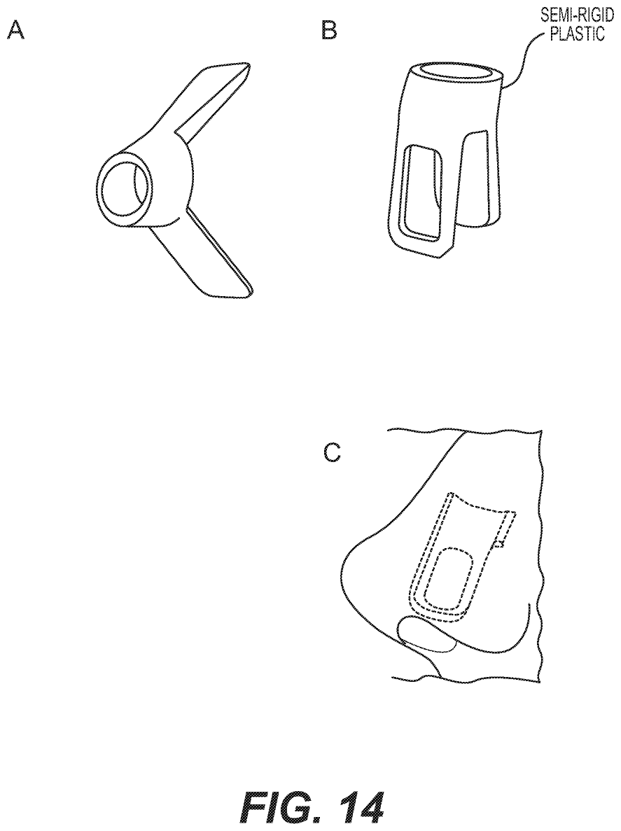

FIG. 14 shows one version of the Elongate-winged dilator. The dilator contains two identical, flexible, curved contact plates or pads connected directly to a circular lumen. FIG. 14A depicts one embodiment of this version of the dilator. FIG. 14B depicts another embodiment of this version of the dilator. When in use, the outward pressure from the supported contact plates or pads relieves nasal obstruction. The contact plates or pads in this design are longer to diffuse the pressure on the septum and the nostril allowing for a more comfortable wear. This concept is essentially a tube that has pads protruding from it that offer some dilation. These pads may have cutouts that provide some ventilation, allow the protrustion of nasal hairs, and allow a connector to be attached to reversibly connect dilators into a symmetric pair or clamp the device to different external portions of the nose. The tubular portion can rest beyond the nostril and the pads can extend further into the nasal passage, or in some embodiments the tubular portion can be inserted first and the pads can rest just beyond the nasal passage. FIG. 14C depicts the correctly positioned dilator in the nasal passage of the user. These embodiments can also use fins that allow for better fit and stability to ensure the device is “aiming” in the direct direction to funnel airflow over the correct portion of the nasal cavity and to keep the device in place even under a sneeze or peak inhalation.

The Alignment Fin Feature with Soft Surface Design

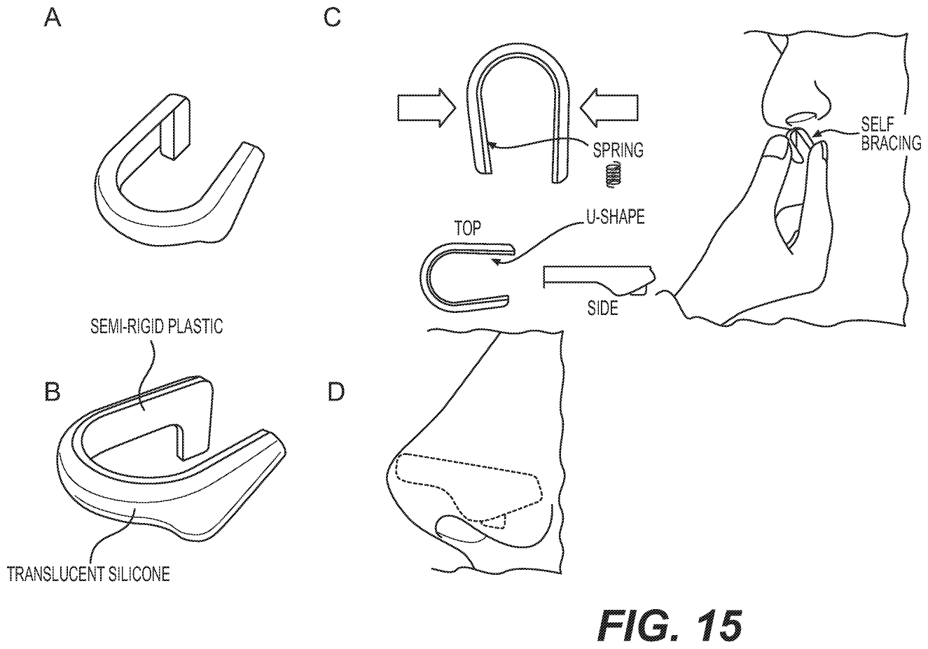

FIG. 15 shows one version of the Alignment fin feature with soft surface dilator. The dilator is U shaped with slightly thicker contact plates on the sides. FIG. 15A depicts one embodiment of this version of the dilator, which is made of semi-rigid plastic. FIG. 15B depicts another embodiment of this version of the dilator, which has a soft silicone gel or other soft material coating the semi-rigid plastic body to diffuse pressure and eliminate sharp corners that would cause discomfort. Each contact plate contains a spring, which allows the device to be squeezed during insertion to allow placement in the nasal airway. The device expands upon release in the correct location in the nasal airway. FIG. 15C depicts the method of inserting the dilator into the nostril of a user. FIG. 15D shows the correctly positioned dilator in the nasal passage of the user, and depicts the design of the fin feature that runs along the run of the nostril and fits into the tip of the nose and the ala for added stability, to aid alignment, and to provide a shallow but hidden feature the user can pull on for easy removal.

Another design contains hinges between the contact plates and the top curved piece of the U shape to facilitate compression and expansion of the device.

In certain designs, the dilator additionally contains one or more hooks or external bands for added stability of the deployed device. The hooks or external bands are reversibly snapped on and off. This allows the user to switch the device between “discreet” mode and “sleep” and/or “athletic” modes.

The Semi-Tubular Design 1

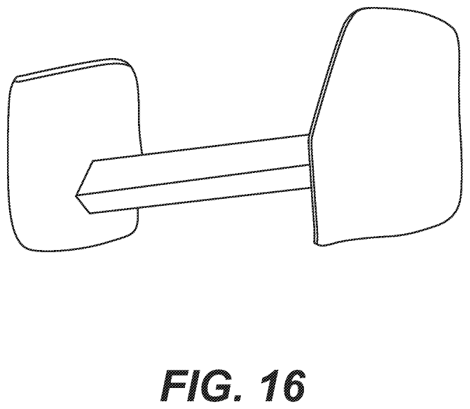

FIG. 16 shows one version of the semi-tubular dilator. The dilator contains two contact plates connected by a single straight, flexible cross-bridge. The septal plate is larger to diffuse the pressure along the septum. The device is squeezed during insertion to allow placement in the nasal airway. The device expands upon release in the correct location in the nasal airway.

In certain designs, the dilator additionally contains one or more hooks or external bands for added stability of the deployed device. The hooks or external bands are reversibly snapped on and off. This allows the user to switch the device between “discreet” mode and “sleep” and/or “athletic” modes.

The Semi-Tubular Design 2

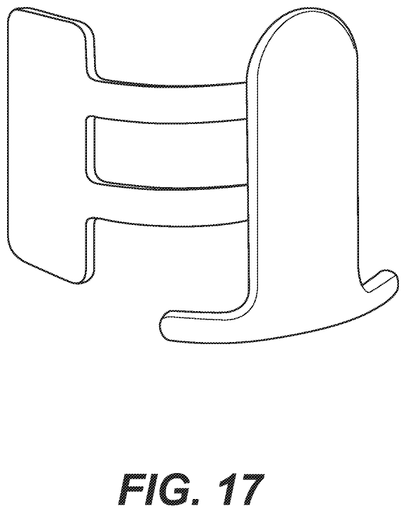

FIG. 17 shows another version of the semi-tubular dilator. The dilator contains two contact plates connected by a two curved, flexible cross-bridges. The septal plate is larger to diffuse the pressure along the septum. The device is squeezed during insertion to allow placement in the nasal airway. The device expands upon release in the correct location in the nasal airway.

In certain designs, the dilator additionally contains one or more hooks or external bands for added stability of the deployed device. The hooks or external bands are reversibly snapped on and off. This allows the user to switch the device between “discreet” mode and “sleep” and/or “athletic” modes.

The Semi-Tubular Design 3

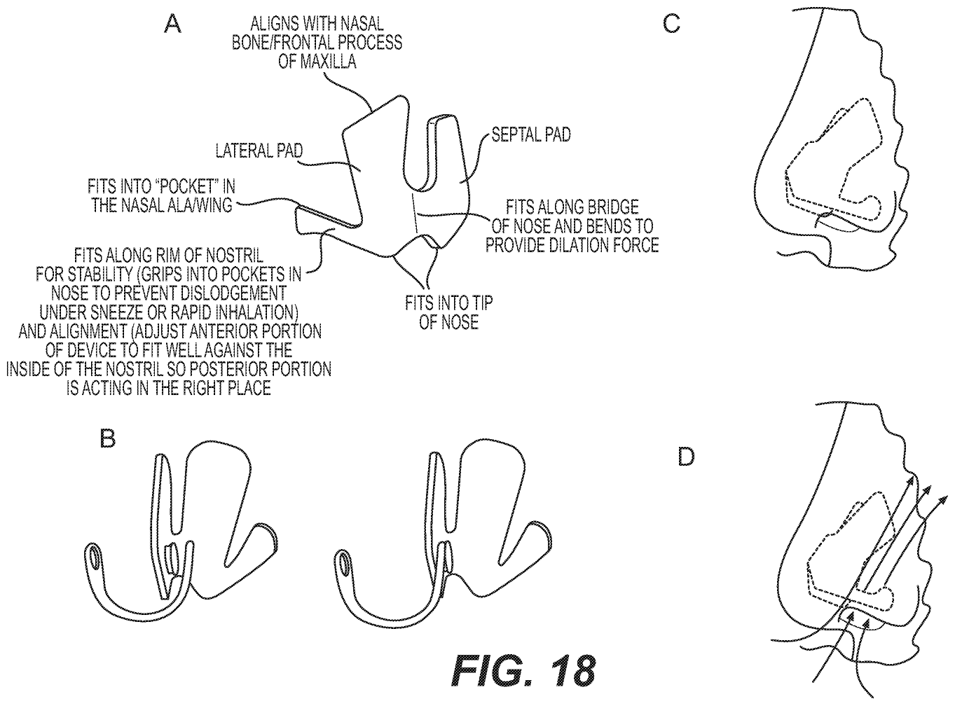

FIG. 18 shows yet another version of the semi-tubular dilator. FIG. 18A depicts the essential features of the dilator. The dilator contains two contact plates (septal pad and lateral pad), a cross-bridge, and an arm. When in use, the septal pad rests against the septum and the lateral pad rests against the lateral nasal wall. The cross-bridge, which is the portion of the dilator in between the pads, fits along the bridge of the nose and bends to provide dilation force. The tip of the lateral pad aligns with the nasal bone/frontal process of maxima. The arm fits into a pocket in the nasal ala. The arm fits along rim of nostril for stability (grips into pockets in the nose to prevent dislodgment under sneeze or rapid inhalation) and alignment (adjust anterior portion of the device to fit well against the inside of the nostril so posterior portion is acting in the right place). The device is squeezed during insertion to allow placement in the nasal airway. The device expands upon release in the correct location in the nasal airway. FIG. 18C depicts the correctly positioned dilator in the nasal passage of a user. The device fits along the bridge of the nose in the crease between the septum and the lateral wall. The ends of the device align with the nasal bone. The prong that runs along the rim of the nostril and fits into the ala provides stability by locking the device into position and aiding alignment and easy removal without a tool. The front of the device fits into the top of the nose for added stability. By fitting into these grooves and spaces in the nose, the device naturally aligns itself correctly and is prevented from rotating, slipping, and becoming dislodged under the force of a sneeze or rapid inhalation. FIG. 18D depicts the airflow through the correctly positioned dilator.

In certain designs, the dilator additionally contains one or more hooks or external bands for added stability of the deployed device. The hooks or external bands are reversibly snapped on and off. This allows the user to switch the device between “discreet” mode and “sleep” and/or “athletic” modes. FIG. 18B a removable septum connector attached to the dilator. The dilator has a button the flexible connector wraps around to lock in place.

The Alignment Fin Feature with Openings Design

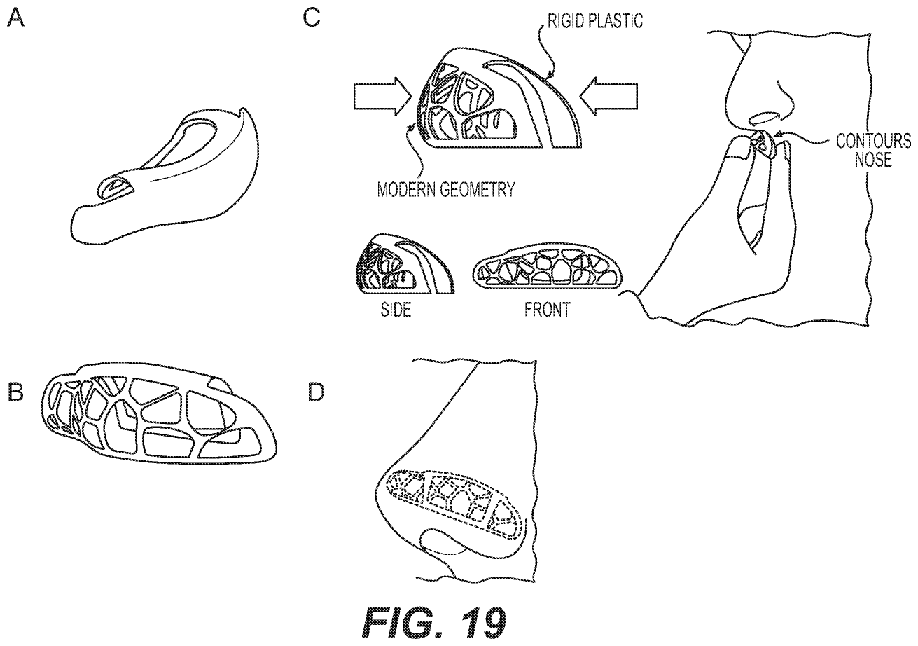

FIG. 19 shows the Alignment fin feature with openings dilator. FIG. 19A depicts one embodiment of this version of the dilator. The dilator is made of a single piece that is shaped to conform to the nasal passage. The septal plate portion is larger to diffuse the pressure along the septum. FIG. 19B depicts another embodiment of this version of the dilator. The contours of this device are more irregular and designed to fit into a particular nose. These stability features could be customized for enhanced fit and comfort. The tessellated pattern is an aesthetic component with a dual function of providing space into which natural nasal hairs can protrude. This allows the dilator to function without disrupting the nose's natural function of filtering the air. The device is squeezed during insertion to allow placement in the nasal airway. The device expands upon release in the correct location in the nasal airway. FIG. 19C depicts the method of inserting the dilator into the nostril of a user. FIG. 19D shows the correctly positioned dilator in the nasal passage of the user, and depicts the design of the fin feature that runs along the run of the nostril and fits into the tip of the nose and the nasal ala for added stability, to aid alignment, and to provide a shallow but hidden feature the user can pull on for easy removal. In certain designs, the dilator additionally contains one or more hooks or external bands for added stability of the deployed device. The hooks or external bands are reversibly snapped on and off. This allows the user to switch the device between “discreet” mode and “sleep” and/or “athletic” modes.

The Notched Nose Cone Design

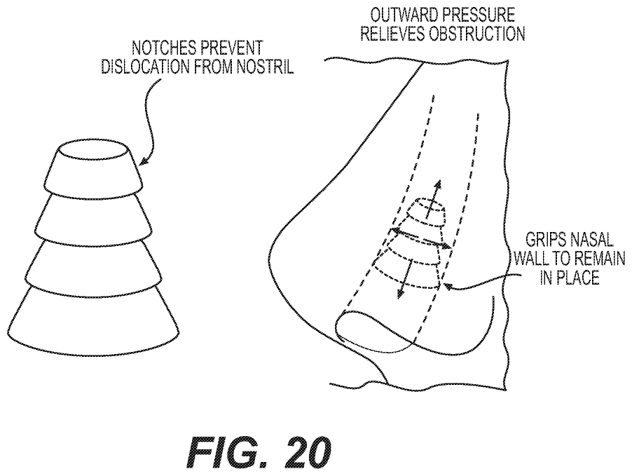

FIG. 20 shows the notched nose cone dilator. The dilator is made of biocompatible silicone. The dilator is conical-shaped, has a notched surface, and a central hollow cavity for airflow that serves as the lumen. The notches prevent the dilator from being displaced from the nostril when in use. When inserted in the nasal passage, the notches on the dilator grip the nasal wall to keep the dilator in place. The outward pressure generated by the correctly positioned dilator relieves nasal obstruction.

The Inverted Notched Nose Cone Design

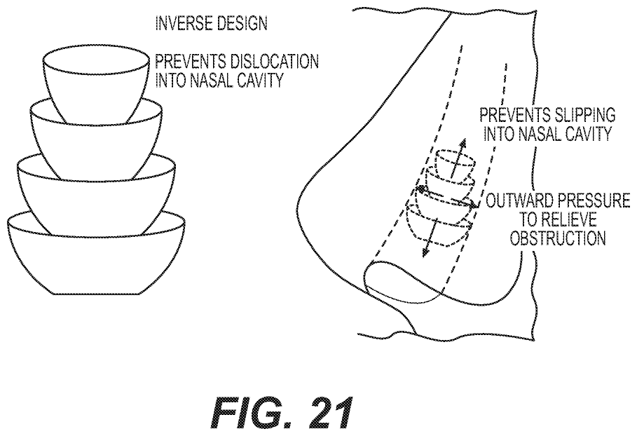

FIG. 21 shows the inverted notched nose cone dilator. The dilator is made of biocompatible silicone. The dilator is conical-shaped, has an inverse notched surface, and a central hollow cavity for airflow that serves as the lumen. The dilator essentially has an inverse design to that of the notched nose cone dilator. When inserted in the nasal passage, the inverse notches on the dilator grip the nasal wall to keep the dilator in place and further prevent the dilator from slipping into the nasal cavity. The outward pressure generated by the correctly positioned dilator relieves nasal obstruction.

The Laminar Flow Design 2

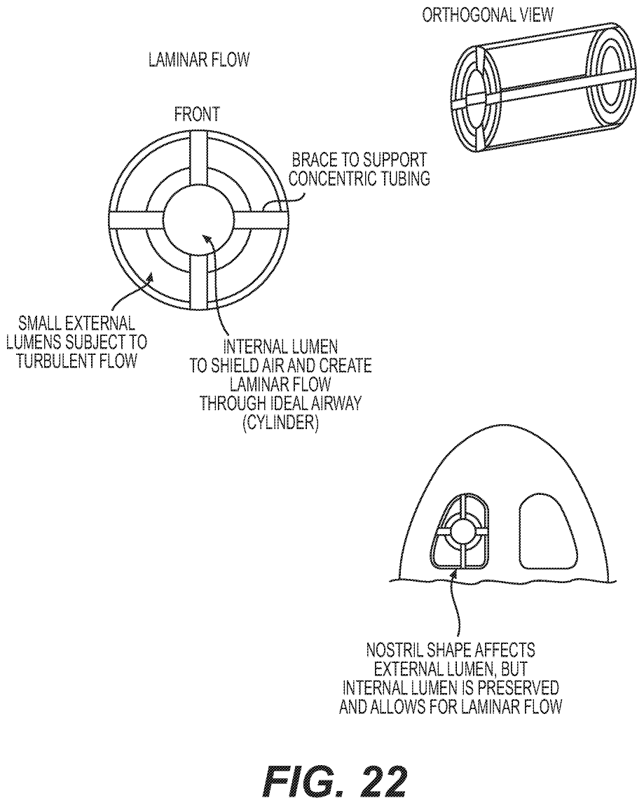

FIG. 22 shows another version of the Laminar Flow dilator. The dilator is cylindrical shaped. It contains two concentric tubings held together by multiple braces. The design creates multiple small external lumens at the each end of the device that are subject to turbulent airflow, and an internal lumen to shield air and create laminar flow through ideal airway. When inserted into the nostril, the nostril shape affects the external lumens, but the internal lumen is preserved and allows for more laminar flow during inhalation to direct more air directly to the lungs.

The Mechanical Tension Rod Design 1

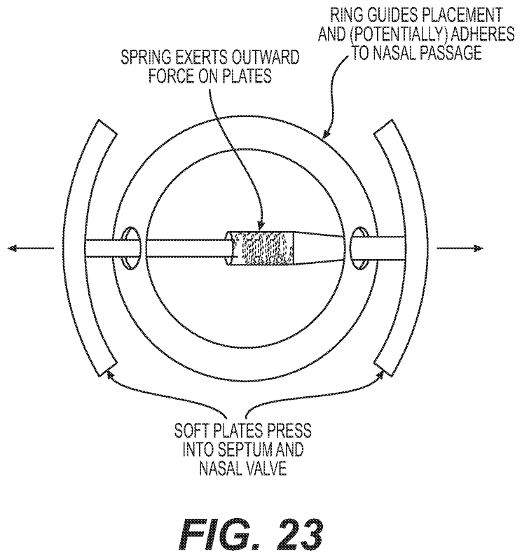

FIG. 23 shows one version of the Mechanical Tension Rod dilator. The dilator consists of two soft nasal contact plates connected by a single straight cross-bridge and a ring structure through which the cross-bridge passes. The ring guides placement of the dilator in the nasal passage and adheres to the nasal passage when the dilator is correctly positioned. When the dilator is in use, the cross-bridge contains a spring that exerts outward force on the contact plates, which press into the septum and nasal valve, thereby relieving nasal obstruction.

The Mechanical Tension Rod Design 2

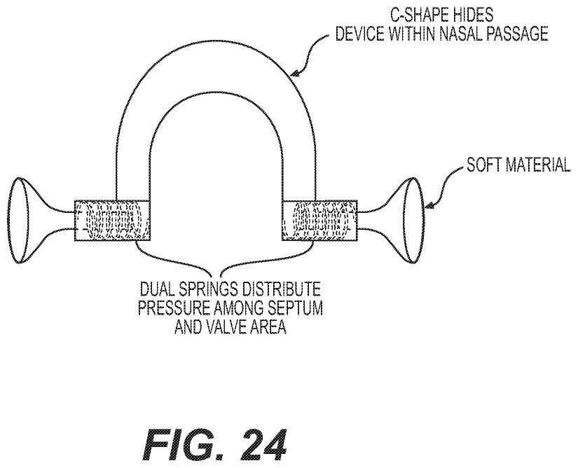

FIG. 24 shows another version of the Mechanical Tension Rod dilator. The dilator consists of two soft nasal contact plates connected by a single C-shaped cross-bridge. The C-shaped cross-bridge hides the dilator within the nasal passage. The contact plates contain dual springs that exert outward force and distribute pressure among the septum and nasal valve area when the dilator is in use, thereby relieving nasal obstruction.

The Mechanical Tension Rod Design 3

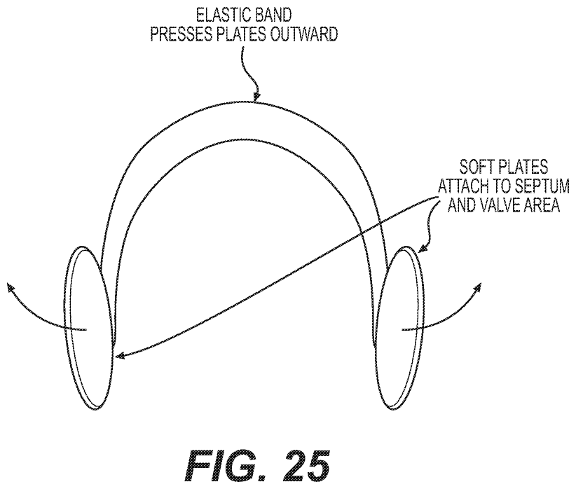

FIG. 25 shows yet another version of the Mechanical Tension Rod dilator. The dilator consists of two soft nasal contact plates connected by a curved elastic band. The device is squeezed during insertion into the nostril to allow placement is the nasal airway. Upon correct positioning and release of the dilator within the nasal airway, the elastic band expands and presses the contact plates outwards, which causes the contact plates to attach to and exert outward force on the septum and nasal valve area, thereby relieving nasal obstruction.

The Mechanical Tension Rod Design 4

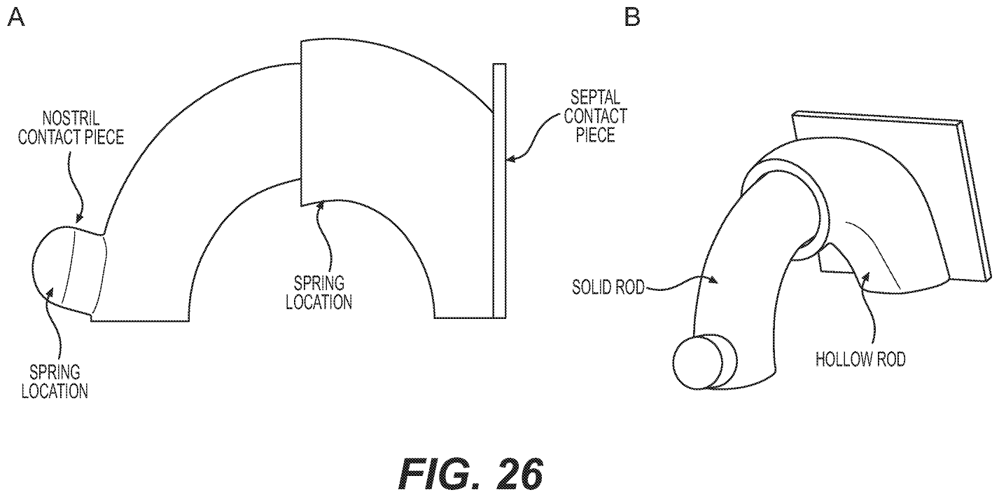

FIG. 26 shows still another version of the Mechanical Tension Rod dilator. FIG. 26A depicts one view of this version of the dilator. FIG. 26B depicts another view of this version of the dilator. The dilator contains a septal contact piece connected to a curved semi-rigid hollow tube and a curved semi-rigid solid rod with a nostril contact piece at its end, which is configured to slide in and out of the hollow tube. There are two compression springs, one within the hollow tube to allow for sizing and one in the nostril contact piece to apply direct pressure on the interior outer wall. The solid rod contains a rigid plate at its other end which contacts the spring within the hollow tube. The nostril contact piece is squeezed to collapse the solid bar into the hollow tube, and the device is positioned within the nasal passage, where the spring within the hollow tube punches the ball-shaped nostril contact piece into the valve area to brace it, thereby relieving nasal obstruction. The septal contact piece has a larger surface area compared to the nasal contact piece to diffuse the pressure on the septum.

The Adjustable Sizing Design 1

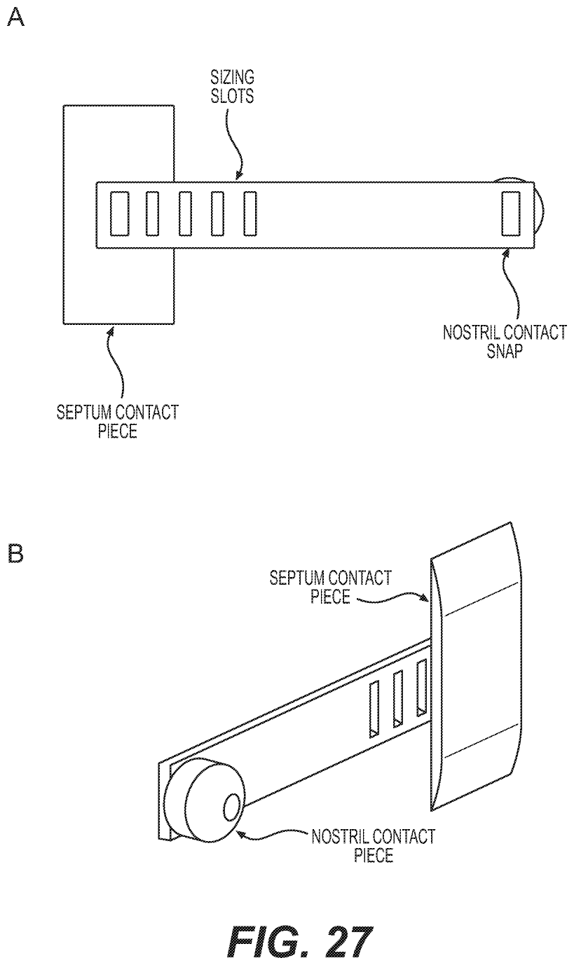

FIG. 27 shows one version of the Adjustable sizing dilator. FIG. 27A depicts one view of this version of the dilator. FIG. 27B depicts another view of this version of the dilator. The dilator contains a septal contact piece and a removable nostril contact piece, connected by a single strip. The strip contains a nostril contact piece snap and several rectangular sizing slots which can accommodate different sized septum contact pieces. The removable nostril contact piece is fixed in place, but different sized contact pieces can be used with the same strip. The septal contact piece can also be exchanged for different sizes and can be moved along the strip to accommodate various sized nostrils. The strip is flexible and is bent to be placed in the nostril, where it then attempts to straighten out, applying pressure on the nostril.

The Adjustable Sizing Design 2

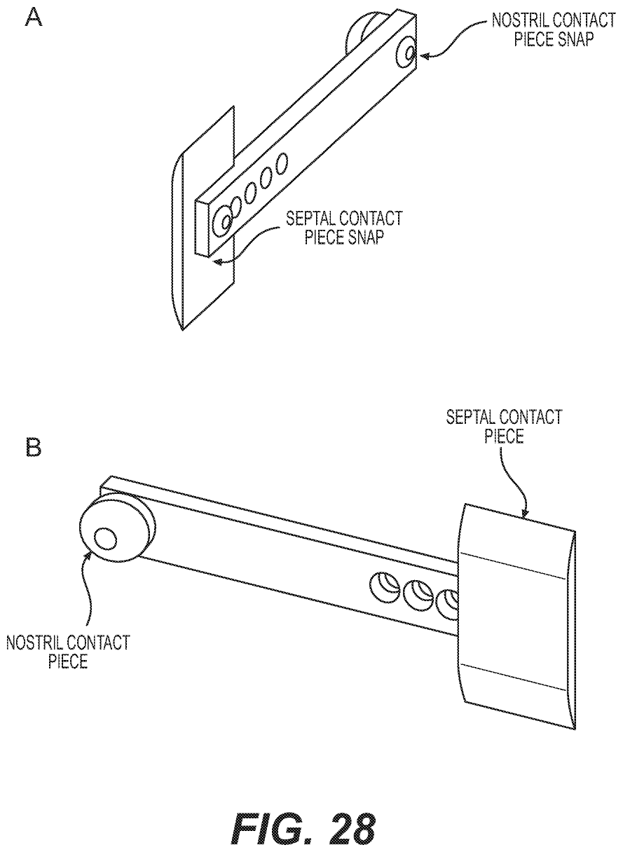

FIG. 28 shows another version of the Adjustable sizing dilator. FIG. 28A depicts one view of this version of the dilator. FIG. 28B depicts another view of this version of the dilator. The dilator contains a septal contact piece and a removable nostril contact piece, connected by a single strip. The strip contains a nostril contact piece snap and several circular sizing slots which can accommodate different sized septum contact pieces. The removable nostril contact piece is fixed in place, but different sized contact pieces can be used with the same strip. The septal contact piece can also be exchanged for different sizes and can be moved along the strip to accommodate various sized nostrils. The strip is flexible and is bent to be placed in the nostril, where it then attempts to straighten out, applying pressure on the nostril.

The Curved Strip Design

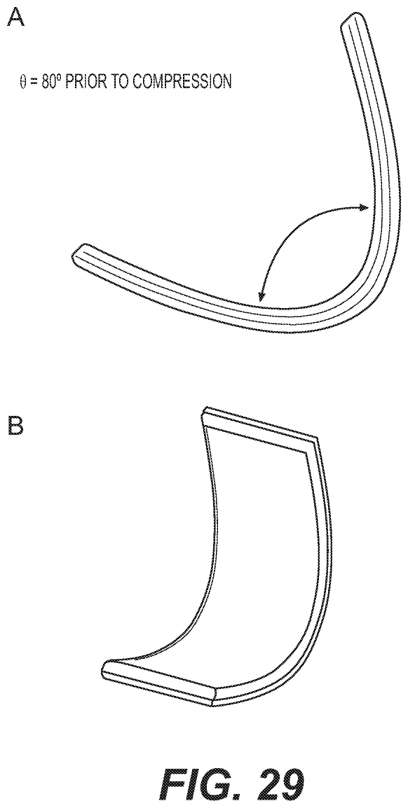

FIG. 29 shows the Curved strip dilator. FIG. 29A depicts one view of the dilator. FIG. 29B depicts another view of the dilator. The dilator is places on the nostril side of the nose and runs along the inside of the nostril to the septum. The angle of the Curved strip dilator is compressed to insert the device into the nose and the force from the device expanding applies pressure on the nostril to open up the nasal valve.

The Curved Strip Dilator with Septal and Lateral Pads Design

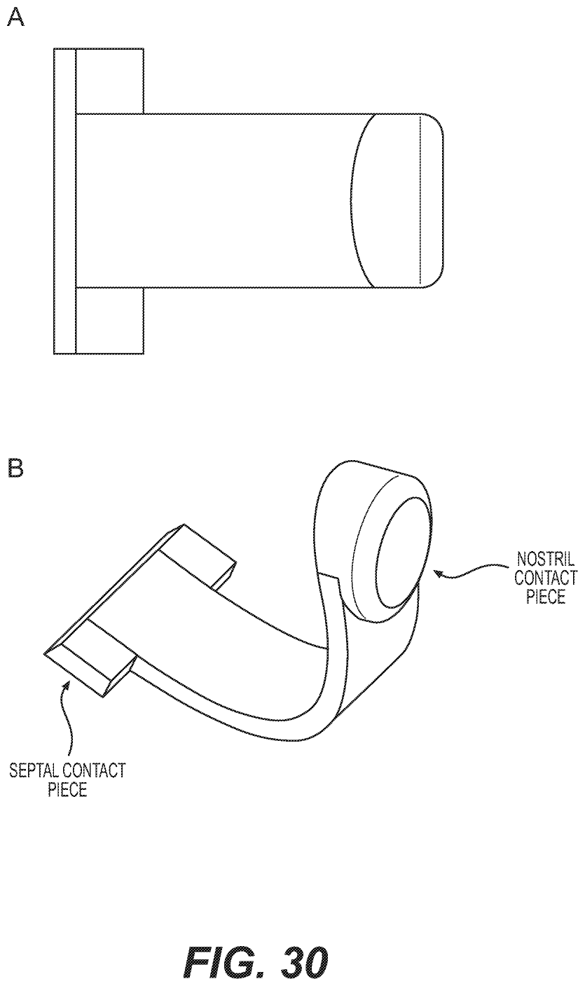

FIG. 30 shows the Curved strip dilator with septal and lateral pads dilator. FIG. 30A depicts one view of the dilator. FIG. 30B depicts another view of the dilator. The dilator contains a septal contact piece and a nostril contact piece connected by a single flexible strip. The flexible strip is bent to be placed in the nostril, where it then attempts to straighten out, applying pressure on the nostril. The dilator has a large surface area for septum contact which allows for decreased pressure on the septum. The nostril contact plate piece allows for more concentrated pressure at the correct place to open up the nasal valve.

The Ratcheting Curved Strip Design

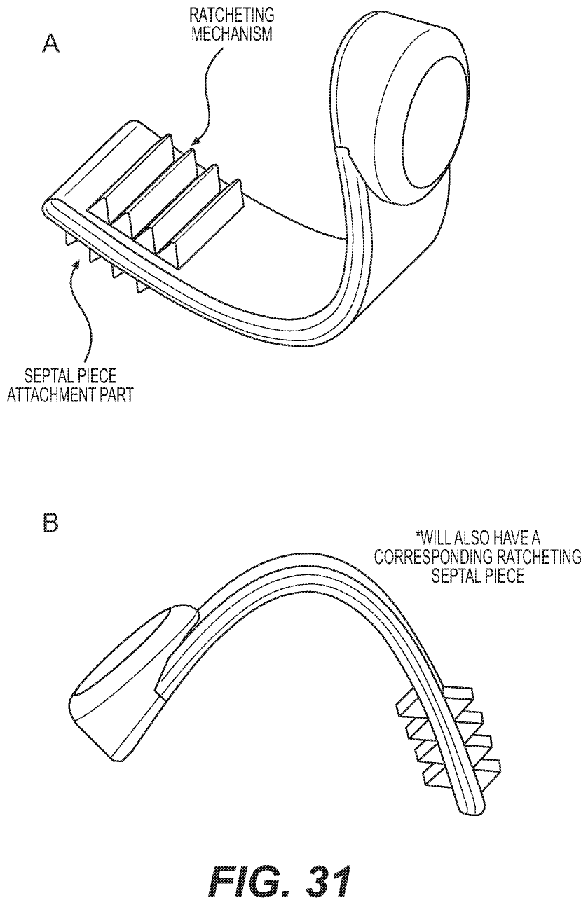

FIG. 31 shows the Ratcheting curved strip dilator. FIG. 31A depicts one view of the dilator. FIG. 31B depicts another view of the dilator. The dilator contains a nostril contact piece connected to a single flexible strip which contains a ratcheting mechanism at its other end. The ratcheting mechanism functions as a septal piece attachment part. A corresponding ratcheting septal piece attaches to the flexible strip via the septal piece attachment part. The ratcheting mechanism allows for adjustment in the placement of the septal piece. The flexible strip is bent to be placed in the nostril, where it then attempts to straighten out, applying pressure on the nostril. The nostril contact plate piece allows for more concentrated pressure at the correct place to open up the nasal valve.

The Semi-Tubular/Modified Triangular Wedge Design

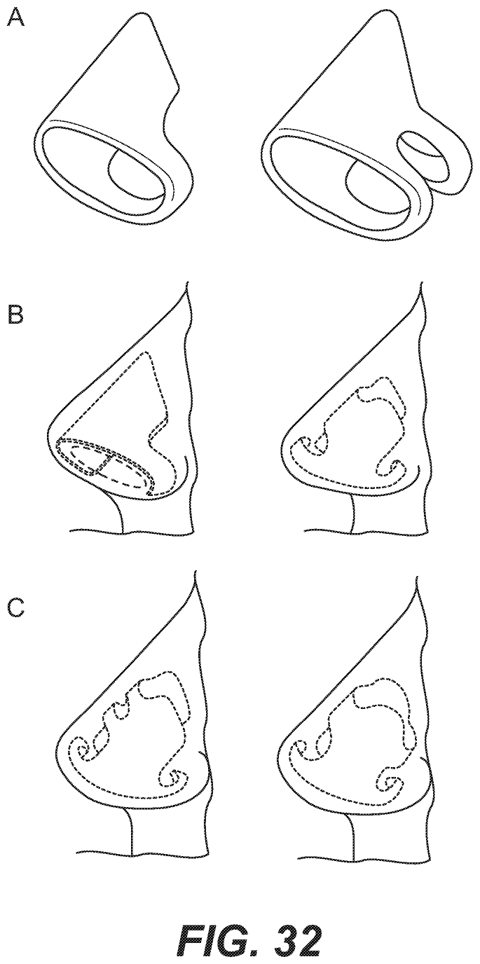

FIG. 32 depicts one embodiment of the semi-tubular/modified Triangular wedge dilator. FIG. 32A depicts one view of the dilator. The dilator can be connected into full tubes at certain points to aid provide extra mechanical support and potentially provide compatibility with a certain type of applicator tool.

FIG. 32B depicts the correctly positioned dilator in the nasal passage of a user. Left: upturned nose. Nostril is shown with dashed oval. The tip of the device fits into the pocket at the tip of the nose. The device has a fin that fits into the ala at the side of the nose. The bridge of the device fits along the bridge of the nose. These features are entirely or partially hidden by the sill beyond the nostril, depending on the individual's anatomy. These features provide stability against the pressure of a sneeze or strong inhalation, aid aid aligment (lining the proximal portion of the device with the nostril ensures the distal portion of the device is positioned properly. The device extends back into the nasal passage and dilates at the internal nasal valve, supporting the region commonly tested in the Cottle manuever and where key rhinoplasty grafts are placed to support the internal nasal valve area. Right: downturned nose. This is a slightly modified version of the concept with greater separation between the septal and lateral pads, and the fin protrudes from the lateral pad into the tip of the nose for stability.

FIG. 32C depicts a modified version of the dilator correctly positioned in the nasal passage of a user. Left: multiple bands run along the bridge of the device for added flexibility. Right: the bottom portion of the device has a band as well, creating a full tube.

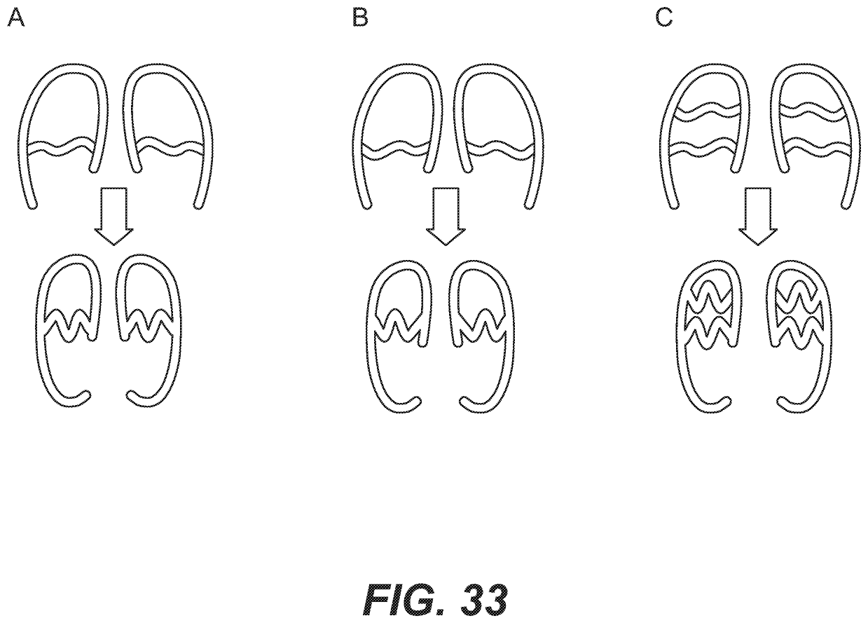

FIG. 33 depicts that the dilator may be connected into full tubes at certain points to aid provide extra mechanical support and potentially provide compatibility with a certain type of applicator tool. These cross-bridges may be simple curved shapes or have a more complex curvature as shown here that reduces the dilation force slightly for improved comfort and causes the compression to occur without buckling or unpredictable bending. FIG. 33A: cross-bridges bend downward. FIG. 33B: cross-bridges bend upward. FIG. 33C: two pairs of cross-bridges bend away from each other to produce a lumen.

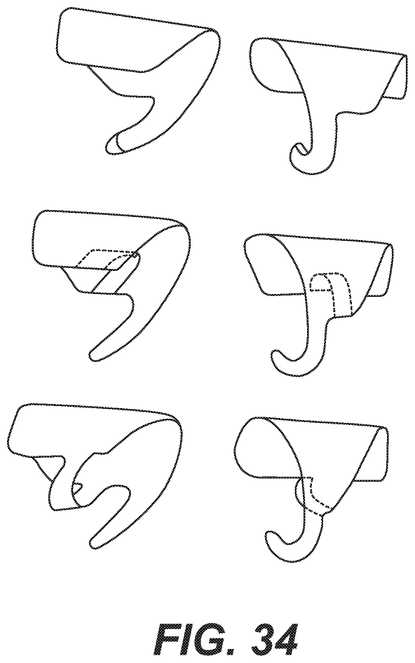

FIG. 34 depicts that the semi-tubular/modified Triangular wedge dilator may be connected into full tubes at certain points to aid provide extra mechanical support and potentially provide compatibility with a certain type of applicator tool. FIG. 34A: normal semi-tubular/modified Triangular wedge dilator with no cross-bridges. FIG. 34B: semi-tubular/modified Triangular wedge dilator with upturned cross-bridges (parallel to the curvature of the device as a whole). FIG. 34C: semi-tubular/modified Triangular wedge dilator with downturned cross-bridges (essentially turning the semi-tube into a full tube.

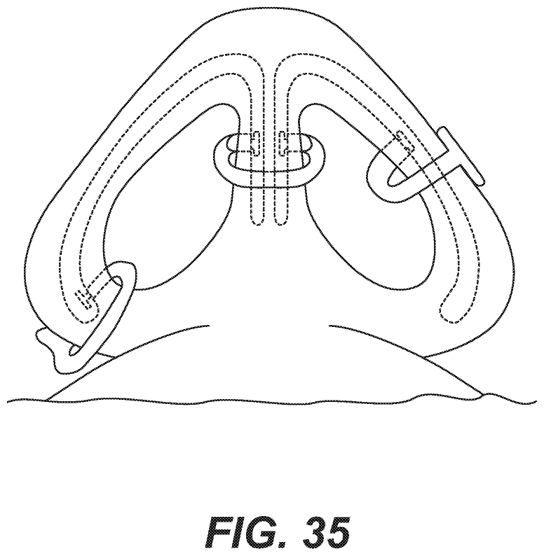

FIG. 35 shows the bottom view of the nose with the semi-tubular/modified Triangular wedge dilator. The figure demonstrates the different places a removable connector between dilators or clamps attaching the dilator to the surface of the nose could be positioned. These surfaces are, e.g., the septum (connecting the dilators in a symmetric pair and potentially clamping the device to the septum), the lateral wall (does not connect the devices into a pair but does provide stability) or the nasal ala (a slightly more discreet clamping option since the external attachment would be partially hidden in the crease between the nose and the cheek).

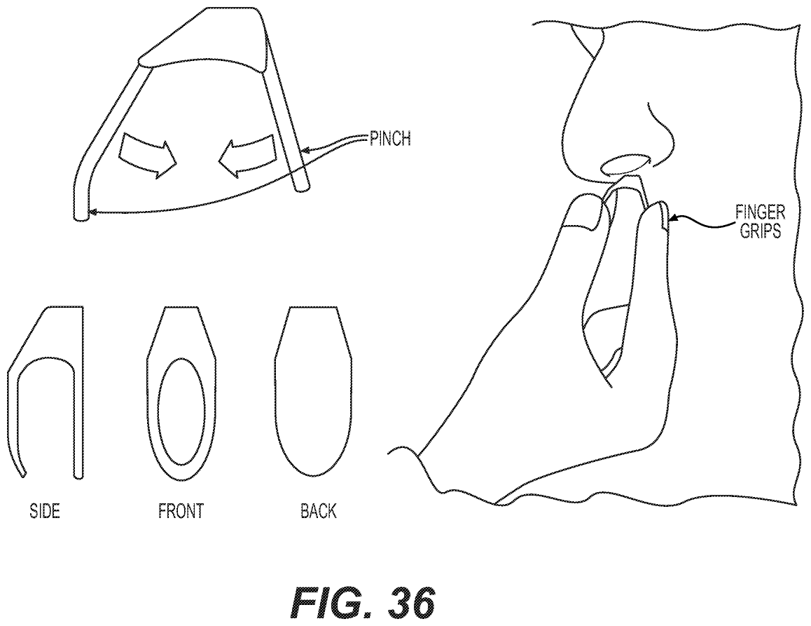

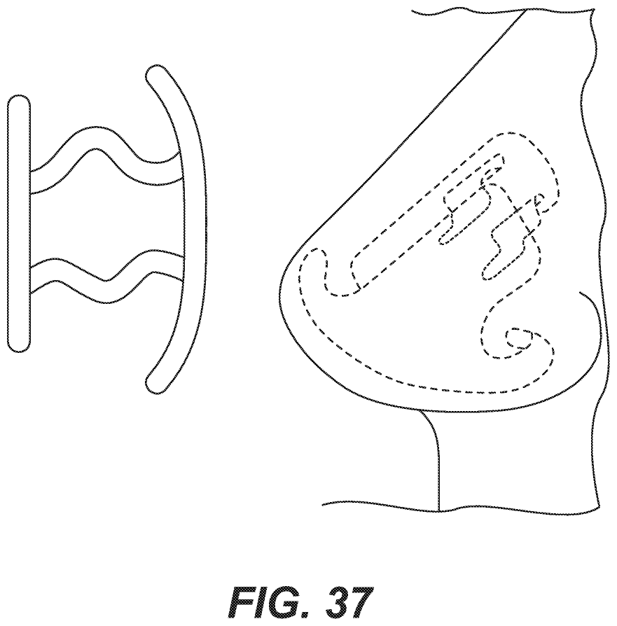

FIGS. 36 and 37 show other embodiments of the semi-tubular/modified Triangular wedge dilator and depict the method of inserting the respective dilators into the nostril of a user.

The Triangular Lumen Design

This version of the triangular lumen dilator contains an outer, flexible, ring and a rigid inner triangular support. When in use, the outward pressure from the supported rings relieves nasal obstruction.

The Magnetic Repulsion Design

The magnetic repulsion dilator contains magnetic pieces that attach to the interior outer wall and the septum. The magnetic pieces are oriented in a manner such that magnets placed in the same nostril repulse each other, whereas magnetic placed in different nostrils on the two sides of the septal wall attract each other. The repulsive forces between the magnets placed within the same nostril force open the nostril and open the nasal valve. Additionally, the attractive forces between the magnets placed in different nostrils on the two sides of the septal wall straighten out a deviated septum. This design can also be used just for opening the nasal valve and not for straightening the deviated septum.

The Magnetic Applicator Design

The magnetic applicator dilator contains a ring-shaped magnetic device that is moved into the correct position in the nasal passage by a magnet which is placed externally on the nostril to attract the dilator.

The Custom Nasal “Invisalign” Design

The “invisalign” dilator contains custom “invisalign” pieces that are inserted to correct nasal anatomy. Over time, new nasal “invisaligns” are made to continue to correct the nasal anatomy. Final nasal “invisalign” finishes correction and relieves nasal obstruction.

The Custom Mold Design

The Custom Mold dilator contains a custom-sized insert to relieve nasal obstruction. The molds are made for each user's nasal passage to allow for custom-sized devices. The molds can be made in an outpatient clinic, or the device itself could be moldable and shaped by the user.

The Ratcheting Ring Design

The Ratcheting Ring dilator contains a ratcheting flexible ring with notches to allow for custom sizing and a receiver for excess. The contracted ratcheting ring expands when inserted into an obstructed nasal airway and supports the nasal valve to relieve nasal obstruction.

The Nasal Cone Screw Design

The Nasal Cone Screw dilator contains a screw made of strong, flexible plastic and score marks to allow for easy “snap off” to remove the excess material once the dilator is properly inserted into the nasal passage. As the dilator is twisted into the nasal passage, it generates an outward pressure, which begins to open the nasal airway. The dilator is twisted into the nasal passage until the nostril is appropriately opened. Then the excess, wider portion of the nasal cone screw can be snapped off to allow for custom sizing.

The Nose Opening Applicator Design

The Nose Opening Applicator dilator is a conical shaped device with a circular lumen that runs through its center. As the dilator is twisted into the nasal passage, it generates an outward pressure, which pulls the nostril open and further allows the insertion of the device. The sides of the dilator contain hooks to grip the nostril edges and keep the dilator in the correct position.

The Hoberman Ring Design

The Hoberman Ring dilator contains an expandable ring made of flexible plastic and score marks to mold to the nasal shape when in use. The ring contains multiple joints to allow for movement of the parts. The dilator is inserted in to the nostril in a compressed form. Once correctly positioned, the ring expands to custom sizes and relieves nasal obstruction.

The Ring with Plates Design

The Ring with Plates dilator contains a rigid ring to exert outward pressure, and two flexible contact plates to adhere to the nostril and septum. The deflated dilator is inserted into the obstructed nostril and inflated. The ring is pinched to collapse the device and allow it to be inserted in the nasal passage. Upon correct positioning and release of the device, the ring expands. Nasal obstruction is relieved from outward pressure from the ring.

The Inflatable Ring with Rigid Exterior Design

The Inflatable Ring with Rigid Exterior dilator contains an inner silicon balloon and an outer silicon balloon, which contain on its surface multiple separated rigid plastic plates with grips. The deflated dilator is inserted into the obstructed nostril and inflated. The area between the inner and outer balloon fills with air and inflates the dilator allowing the rigid plates to grip the nasal and septal walls. Outward pressure from the inflated dilator relieves nasal obstruction.

The Inflatable Ring with Rigid Interior Design

The Inflatable Ring with Rigid Interior dilator contains an inner silicon balloon with multiple overlapping rigid plastic plates on its surface, and an outer silicon balloon. The deflated dilator is inserted into the obstructed nostril and inflated. The area between the inner and outer balloon fills with air and inflates the dilator causing the overlapping plates to become separated. Outward pressure from the inflated dilator relieves nasal obstruction.

The Check Valve Design

This design increases the nasal valve angle by modifying the pressure differential in order to use normal breathing airflow patterns to open the nasal valve. The Check Valve dilator contains a hollow tube which has inserted in it a funnel shaped tube containing a check valve that is configured to open only when the user in breathing in. Once the dilator is correctly positioned in the nasal passage, breathing in by the user exerts pressure on the check valve, pushing it out and allowing for an increased nasal valve angle.

The Butterfly Valve Design

This design imitates a CPAP (Continuous Positive Airway Pressure) machine in its action. The Butterfly Valve dilator is a ring-shaped device that contains a spinning disk, which only allows airflow in one direction. When in use, breathing in by the user exerts pressure on the valve, pushing it out and opening up the nasal valve.

The Strong Internal Skelton Design

The Strong Internal Skeleton dilator contains a soft polymer outer surface that is comfortable against skin and a strong, flexible rod, which is embedded within the soft polymer. The dilator is bent to be placed in the nostril, where the flexible rod attempts to straighten out, applying pressure on the nostril.

The Slow Release Antihistamine Design

This design increases the nasal valve angle using chemical methods. The Slow Release Antihistamine dilator contains a ring containing a hydrogel with antihistamine. When the dilator is placed in the nostril, antihistamine is slowly released over time into the nasal airway to help alleviate obstruction.

The Extraction String Design

The Extraction String dilator contains a ring, which can be inserted in the nasal passage to relieve obstruction. A string is attached to the ring and hangs down the inside of the nose to allow for easy extraction of the device. Alternatively, a tweezer-like tool is used to remove the device from the nostril.

The Mesh Filter Design

The Mesh Filter dilator contains a ring attached to a mesh-like filter, which can be inserted in the nasal passage to relieve obstruction. The filter allows for airflow both into and out of the nasal passage, but prevents particles from entering the body.

The Microfiber Filter Design

The Microfiber Filter dilator contains a ring with attached microfibers, which can be inserted in the nasal passage to relieve obstruction. The microfibers allow for airflow both into and out of the nasal passage while helping with filtration of the air passing through the device.

The Tesla Filter Design

The Tesla Filter dilator contains a cylindrical hollow tube containing loops of Tesla filters, which cause high turbulence and therefore trap air. The Tesla filters, which are used in microfluidics, allow the air to come in during inhalation without entirely blocking exhalation, thereby creating a pressure differential that causes the nasal valve to open.

The Feather Filter Design

The Feather Filter dilator contains a cylindrical hollow tube containing internal fibers or feathers. The fibers/filters allow air to come in during inhalation. During exhalation, however, the air lifts up the fibers or feathers blocking exhalation. This creates a pressure differential that causes the nasal valve to open.

The Nose Tent Design

The Nose Tent dilator contains two soft contact plates for comfort, connected by a single flexible rod. The flexible rod is not collapsible and applies outward pressure on nostril to relieve nasal obstruction.

The Mechanical Tension Rod Design 5

This version of the tension rod dilator contains two soft nasal contact plates that conform to the shape of the nasal passage connected by a tube structure. The tube structure consists of an outer hollow tube that contains a spring and an inner solid tube that presses against the spring and is loosely fit to allow sliding. The contact plates are squeezed to compress the spring and collapse the dilator. The collapsed dilator is positioned within the nasal passage, where the spring expands to bridge the septum and nasal valve area to counteract nasal valve collapse and relieve nasal obstruction. The hollow tube also contains notches to prevent separation of the contact plates. The soft contact plates conform to the nasal wall and provide large surface areas to spread the outward force.

The Mechanical Tension Rod Design 6

This version of the tension rod dilator contains two soft nasal contact plates connected by a single curved cross-bridge. The cross-bridge contains a torsional spring that spreads the contact plates. When the dilator is in use, the contact plates adhere to the septum and nasal valve and exert outward force, thereby relieving nasal obstruction.

The Mechanical Tension Rod Design 7

This version of the dilator contains a septal contact piece connected to a straight semi-rigid hollow tube, to which is fitted a compression spring, and a semi-rigid solid rod fitted with a ball-shaped nostril contact piece. The solid rod is configured to slide in and out of the hollow tube. The nostril contact piece is squeezed to collapse the solid bar into the hollow tube, and the device is positioned within the nasal passage, where the spring within the hollow tube punches the ball-shaped nostril contact piece into the valve area to brace it, thereby relieving nasal obstruction.

The Mechanical Tension Rod Design 8

This version of the dilator contains a straight semi-rigid hollow tube, to which is fitted a compression spring, and a semi-rigid solid rod fitted with a soft tab and a rubber stopper. The solid rod is configured to slide in and out of the hollow tube. The soft tab is squeezed to collapse the solid bar into the hollow tube, and the device is positioned within the nasal passage, where the spring within the hollow tube expands to push the soft tab against the interior outer wall. This creates an outward pressure causing the nasal valve to open and relieving nasal obstruction. The rubber stopper simultaneously wedges into the hollow tube creating a stable, locked position for the expanded device.

The Airplane Wing Design

The Airplane Wing dilator is a cylindrical shaped dilator containing two rigid semi-circular airfoils with semi-circular openings connected by two highly elastic strips. The flow across the airfoils creates a lift according to Bernoulli Effect, pushing out the nasal valve.

The Nose “Sail” Design

The Nose “Sail” dilator is shaped like half a contact lens and contains a conical rough membrane that fits into one half of the nostril. The membrane creates turbulent flow of air during inhalation, thereby causing the nasal valve to open.

The Wrap Design

The Wrap dilator is made of a soft, flexible, biocompatible material and contains a septum pad and a lateral pad connected by two straight flexible cross-bridges. The septum pad is appropriately curved to fit around the lateral pad. The device is scrolled up by wrapping the septum pad around the lateral pad. The scrolled up device unfurls when deployed in the nasal passage, thereby causing the nasal valve to open.

The device is folded into a semi-tube that fits along the bridge of the nose in the crease where the nasal septum and sidewall meet. For stability and more intuitive positioning, the “front” of the device is designed to fit into the pocket at the tip of the nose and has an arm that runs along the rim on the side of the nose just beyond the nostril. The “back” of the device is angled to fit up against the nasal bone. The design nicely mimics the appearance and functionality of the natural nasal cartilage and directly targets the internal nasal valve.

### EXAMPLES

### Example 1

### Testing Airflow Using the Nasal Dilation Device

The present dilator was tested for airflow. 8 subjects at rest breathed inward as forcefully as possible through a mask designed to isolate nasal breathing and a transducer (Biopac) that measures airflow and tidal volume. In the Biopac software the inventors determined the peak nasal inspiratory flow (PNIF), a metric that has been shown to correlate with the severity of nasal obstruction symptoms. PNIF was recorded with no dilator in place (unaided breathing to serve as a control), prototypes of the present dilator, and commercially available nasal dilators, including Breathe Right strips, Sleep Right nasal breathing aid, and Nozovent.

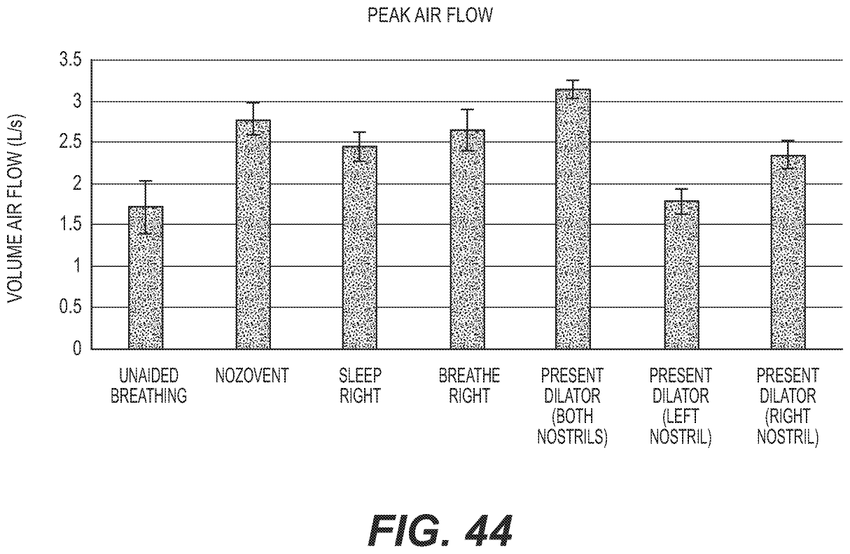

Conclusion: As shown in FIG. 44, peak airflow with the present device is higher than all other tested competitors when used in both nostrils.

1. A nasal passage dilator for positioning internally within a single nostril, the nostril having a septum, an internal nasal passage, and an interior outer wall surface opposite the septum, the dilator comprising:
a. a first contact plate configured to contact and apply an outward force to the interior outer wall surface when the dilator is positioned in the nasal passage;
b. a second contact plate configured to contact and apply an outward force to the septum when the dilator is positioned in the nasal passage;
c. at least one cross-bridge connecting the first contact plate to the second plate; and
d. a lumen for air flow, wherein the lumen is a hollow space bordered by the first and second contact plates and the at least one cross-bridge,
wherein the dilator is configured to rest entirely within the nasal passage of the single nostril.

2-106. (canceled)
